(19) United States
(12) Patent Application Publication
(10) Pub. No.: US 2022/0023668 A1
(43) Pub. Date: Jan. 27, 2022

(54) ULTRASOUND ANNULAR ARRAY DEVICE FOR NEUROMODULATION

(71) Applicant: Liminal Sciences, Inc., Guilford, CT (US)

(72) Inventors: Kamyar Firouzi, San Jose, CA (US); Yichi Zhang, Mountain View, CA (US); Jose Camara, Saratoga, CA (US); Guillaume David, Palo Alto, CA (US)

(73) Assignee: Liminal Sciences, Inc., Guilford, CT (US)

(21) Appl. No.: 17/381,966

(22) Filed: Jul. 21, 2021

Related U.S. Application Data
(60) Provisional application No. 63/057,648, filed on Jul. 28, 2020.
(60) Provisional application No. 63/054,667, filed on Jul. 21, 2020.

(51) Int. Cl. A61N 7/00

(52) U.S. Cl. CPC A61N 7/00; A61N 2007/0078; A61N 2007/0052; A61N 2007/0026

(57) ABSTRACT

In some aspects, the described systems and methods provide for a device wearable by or attached to a person, comprising at least one annular array transducer configured to provide ultrasound radiation to at least one region of the brain of the person. In some embodiments, the annular array transducer is configured to provide the ultrasound radiation to perform non-invasive neuromodulation or neurostimulation in the at least one region of the brain of the person.

### CROSS REFERENCE TO RELATED APPLICATIONS

This application claims priority under 35 U.S.C. § 119(e) to U.S. Provisional Patent Application Ser. No. 63/054,667, titled “SYSTEMS AND METHODS FOR A NEUROMODULATION DEVICE,” filed Jul. 21, 2020 and U.S. Provisional Patent Application Ser. No. 63/057,648, titled “ULTRASOUND ANNULAR ARRAY DEVICE FOR NEUROMODULATION,” filed Jul. 28, 2020, both of which are hereby incorporated herein by reference in their entireties.

### BACKGROUND

Conventional techniques to transmit ultrasound into the brain are implemented by means of a large-aperture spherical transducer consisting of a very large number of single element transducers transmitting ultrasound beams through the skull. The geometric focus of these transducers is typically limited to the center of the brain, whereas the majority of cancers and neurological disorders, especially metastases, occur along or originate in the periphery of the brain. Moreover, conventional technology is cost-prohibitive which impedes its widespread application for neurological disorders.

### SUMMARY

The inventors have recognized the above shortcomings in the current state of the art and have developed novel devices and techniques to address such deficiencies. In particular, the inventors have developed a novel annular array technology for focusing ultrasound radiation for non-invasive therapy to different regions of the brain, including neuromodulation or neurostimulation applications, with the capability of providing both continuous and/or acute therapy. The therapeutic fields of application can include, but are not limited to, epilepsy and seizure, neurological disorders such as Alzheimer's disease, depression, Parkinson's disease, and multiple sclerosis, tissue ablation for conditions such as tumor and essential tremor, and opening blood brain barrier (BBB) and drug delivery. Other fields of applications can include those of imaging, including elastography, Acoustic Radiation Force Imaging (ARFI), and doppler for measuring tissue motion and/or blood motion.

In some aspects, a device wearable by or attached to a person comprises at least one annular array transducer configured to provide ultrasound radiation to perform non-invasive neuromodulation in at least one region of the brain of the person.

In some embodiments, the device comprises circuitry configured to receive echo data from the annular array transducer and, based on the echo data, correct an amplitude and/or a phase of the ultrasound radiation.

In some embodiments, the annular array transducer is further configured to provide the ultrasound radiation to perform non-invasive neurostimulation in the at least one region of the brain of the person.

In some embodiments, the annular array transducer comprises a plurality of concentric elements, wherein at least one of the plurality of concentric elements is operable to provide the ultrasound radiation.

In some embodiments, each of the plurality of concentric elements have substantially the same surface area.

In some embodiments, each of the plurality of concentric elements have substantially the same width.

In some embodiments, the device comprises circuitry configured to independently drive electrical energy to each element of the plurality of concentric elements.

In some embodiments, a time profile of the electrical energy includes a continuous wave, a quasi-continuous wave, and/or a pulsed wave.

In some embodiments, the device comprises circuitry configured to assign a phase to each element of the plurality of concentric elements, wherein the phase assigned to the element is independent from phases for other elements.

In some embodiments, the circuitry is further configured to assign the phase to each element of the plurality of concentric elements based on a distance from the element to a target focal depth in the at least one region of the brain of the person.

In some embodiments, the circuitry is further configured to adjust the target focal depth by adjusting the phase of one or more elements of the plurality of concentric elements.

In some embodiments, the annular array transducer is fabricated by radially dicing a piezoelectric material into a plurality of concentric elements.

In some embodiments, the piezoelectric material is selected such that the piezoelectric material has a minimal lateral mode coupling.

In some embodiments, the piezoelectric material includes a 1-3 composite, lead metaniobate, a single-crystal piezoelectric material, and/or a composite piezoelectric material.

In some embodiments, the annular array transducer has a center frequency in a range from 200 kHz to 1 MHz.

In some embodiments, the annular array transducer has a diameter range from 1 to 4 inches.

In some embodiments, the annular array transducer has a fractional bandwidth range from 10% to 60% of a center frequency for the annular array transducer.

In some embodiments, the device includes a processor configured to guide the ultrasound radiation to the at least one region of the brain of the person using ultrasound, optoacoustics, photoacoustics, thermo-acoustics, doppler and functional ultrasound, magnetic resonance-based radiation force imaging (RFI), shear wave elastography, magnetic resonance thermometry, functional imaging techniques, electroencephalography, optical tracking, and/or simulation-based guidance.

In some embodiments, the annular array transducer comprises a plurality of concentric segments, each segment of the plurality of concentric segments comprising a plurality of elements along a circumference of the segment, wherein at least one of the plurality of elements is operable to provide the ultrasound radiation.

In some embodiments, the device comprises circuitry configured to independently drive electrical energy to each element of the plurality of elements, of each segment of the plurality of concentric segments.

In some embodiments, a time profile of the electrical energy includes a continuous wave, a quasi-continuous wave, and/or a pulsed wave.

In some embodiments, the device comprises circuitry configured to assign a phase to each element of the plurality of elements, of each segment of the plurality of concentric segments, wherein the phase assigned to the element is independent from phases for other elements.

In some embodiments, the circuitry is further configured to assign the phase to each element of the plurality of elements, of each segment of the plurality of concentric segments, based on a distance from the element to a target focal depth in the at least one region of the brain of the person.

In some embodiments, the circuitry is configured to adjust the target focal depth in up to three dimensions by adjusting the phase of one or more elements of the plurality of elements of each segment of the plurality of concentric segments.

In some embodiments, the annular array transducer is fabricated by radially dicing a piezoelectric material into a plurality of concentric segments and circumferentially dicing each segment of the plurality of concentric segments into a plurality of elements.

In some aspects, a device wearable by or attached to a person for providing non-invasive neuromodulation or neurostimulation to at least one region of the brain of the person, comprises an oscillator, a phase generator coupled to the oscillator, a plurality of power amplifiers coupled to the phase generator, a plurality of tuners, each tuner coupled to a power amplifier of the plurality of power amplifiers, at least one annular array transducer configured to generate ultrasound radiation, each element of the annular array transducer coupled to a tuner of the plurality of tuners, and feedback circuitry coupled to the annular array transducer and the phase generator.

In some embodiments, the feedback circuitry is configured to receive echo data from the annular array transducer and transmit the echo data to the phase generator.

In some embodiments, the phase generator is configured to correct an amplitude and/or a phase of the ultrasound radiation based on the echo data.

In some embodiments, the device includes a processor configured to guide the ultrasound radiation to the at least one region of the brain of the person using ultrasound, optoacoustics, photoacoustics, thermo-acoustics, doppler and functional ultrasound, magnetic resonance-based radiation force imaging (RFI), shear wave elastography, magnetic resonance thermometry, functional imaging techniques, electroencephalography, optical tracking, and/or simulation-based guidance.

In some embodiments, the device includes a processor configured to guide the ultrasound radiation to the at least one region of the brain of the person using portable magnetic resonance imaging having a field strength less than 10 mT, between 10 mT and 0.1 T, or between 0.1 T and 0.2 T.

In some aspects, a method of making a device wearable by or attached to a person for providing non-invasive neuromodulation or neurostimulation to at least one region of the brain of the person comprises providing an oscillator, providing a phase generator coupled to the oscillator, providing a plurality of power amplifiers coupled to the phase generator, providing a plurality of tuners, each tuner coupled to a power amplifier of the plurality of power amplifiers, providing at least one annular array transducer, each element of the annular array transducer coupled to a tuner of the plurality of tuners, and providing feedback circuitry coupled to the annular array transducer and the phase generator.

In some aspects, a method comprises using a device to provide ultrasound radiation to perform non-invasive neuromodulation or neurostimulation in at least one region of the brain of the person, wherein the device comprises an oscillator, a phase generator coupled to the oscillator, a plurality of power amplifiers coupled to the phase generator, a plurality of tuners, each tuner coupled to a power amplifier of the plurality of power amplifiers, at least one annular array transducer configured to generate the ultrasound radiation, each element of the annular array transducer coupled to a tuner of the plurality of tuners, and feedback circuitry coupled to the annular array transducer and the phase generator.

While some aspects and/or embodiments described herein are described with respect to certain brain conditions, these aspects and/or embodiments may be equally applicable to monitoring and/or treating symptoms for any suitable neurological disorder or brain condition. Any limitations of the embodiments described herein are limitations only of those embodiments and are not limitations of any other embodiments described herein.

### BRIEF DESCRIPTION OF THE DRAWINGS

Various aspects and embodiments will be described with reference to the following figures. The figures are not necessarily drawn to scale.

FIG. 1 shows a front view of an annular array transducer, in accordance with some embodiments of the technology described herein.

FIG. 2 shows an exemplary diagram for calculating time and phase delay for each element of an annular array transducer, in accordance with some embodiments of the technology described herein.

FIGS. 3A-3B show exemplary diagrams of aspect ratio considerations for design of an annular array transducer, in accordance with some embodiments of the technology described herein.

FIG. 4 shows exemplary methods for fabricating an annular array transducer, in accordance with some embodiments of the technology described herein.

FIG. 5 shows an exemplary diagram of an annular array transducer integrated with an application specific integrated circuit (ASIC) and housing, in accordance with some embodiments of the technology described herein.

FIG. 6 shows an exemplary block diagram of electronics for driving an annular array transducer, in accordance with some embodiments of the technology described herein.

FIG. 7 shows exemplary diagrams for focusing performance for an 8-channel annular array transducer design with a 3-inch aperture at 500 kHz as a function of F#, in accordance with some embodiments of the technology described herein.

FIG. 8 shows exemplary diagrams for focusing performance for a 16-channel annular array transducer design with a 3-inch aperture at 500 kHz as a function of F#, in accordance with some embodiments of the technology described herein.

FIG. 9 shows exemplary diagrams for performance comparisons between an 8-channel design, a 16-channel design, and a contiguous design for an annular array transducer with a 3-inch aperture at 500 kHz, in accordance with some embodiments of the technology described herein.

FIG. 10 shows exemplary diagrams for performance comparisons between an 8-channel design (top) and a 16-channel design (bottom) for an annular array transducer with a 3-inch aperture at 500 kHz, in accordance with some embodiments of the technology described herein.

FIG. 11 shows exemplary diagrams for focusing performance for an 8-channel annular array transducer design with a 2-inch aperture at 500 kHz as a function of F#, in accordance with some embodiments of the technology described herein.

FIG. 12 shows exemplary diagrams for focusing performance for a 16-channel annular array transducer design with a 2-inch aperture at 500 kHz as a function of F#, in accordance with some embodiments of the technology described herein.

FIG. 13 shows exemplary diagrams for performance comparisons between an 8-channel design (top) and a 16-channel design (bottom) for an annular array transducer with a 2-inch aperture at 500 kHz, in accordance with some embodiments of the technology described herein.

FIG. 14 shows exemplary diagrams for performance comparisons between an 8-channel design, a 16-channel design, and a contiguous design for an annular array transducer with a 2-inch aperture at 500 kHz, in accordance with some embodiments of the technology described herein.

FIG. 15 shows exemplary diagrams for focusing performance for an 8-channel annular array transducer design with a 2-inch aperture at 1 MHz as a function of F#, in accordance with some embodiments of the technology described herein.

FIG. 16 shows exemplary diagrams for focusing performance for a 16-channel annular array transducer design with a 2-inch aperture at 1 MHz as a function of F#, in accordance with some embodiments of the technology described herein.

FIG. 17 shows exemplary diagrams for performance comparisons between an 8-channel design, a 16-channel design, and a contiguous design for an annular array transducer with a 2-inch aperture at 1 MHz, in accordance with some embodiments of the technology described herein.

FIG. 18 shows exemplary diagrams for performance comparisons between an 8-channel design (top) and a 16-channel design (bottom) for an annular array transducer with a 2-inch aperture at 1 MHz, in accordance with some embodiments of the technology described herein.

FIGS. 19A-19G show exemplary diagrams for focusing performance for an 8-channel annular array transducer design with a 3-inch aperture at 500 kHz as a function of F#, in accordance with some embodiments of the technology described herein.

FIG. 20 shows an illustrative embodiment of a fabricated annular array transducer, in accordance with some embodiments of the technology described herein.

FIG. 21 shows an exemplary workflow for a neuromodulation device equipped with guiding capabilities, in accordance with some embodiments of the technology described herein.

FIG. 22 shows another illustrative embodiment of a fabricated annular array transducer, in accordance with some embodiments of the technology described herein.

FIG. 23 shows yet another illustrative embodiment of a fabricated annular array transducer, in accordance with some embodiments of the technology described herein.

FIG. 24 shows yet another illustrative embodiment of a fabricated annular array transducer, in accordance with some embodiments of the technology described herein.

FIG. 25 shows an exemplary workflow for a neuromodulation device equipped with 3D/volumetric scanning capabilities, in accordance with some embodiments of the technology described herein.

FIG. 26 shows an illustrative flow diagram for a process for constructing and deploying a machine learning algorithm, in accordance with some embodiments of the technology described herein.

FIG. 27 shows a convolutional neural network that may be used in conjunction with the described devices and techniques, in accordance with some embodiments of the technology described herein.

FIG. 28 shows a block diagram of an illustrative computer system that may be used in implementing some embodiments of the technology described herein.

### DETAILED DESCRIPTION

Ultrasonic neuromodulation and neurostimulation are non-invasive technologies that utilize low-intensity focused ultrasound (LIFU) to modulate or stimulate neural activity in specific areas of the brain. Neurons in the brain are sensitive to ultrasound. If ultrasound radiation is applied to a region of the brain with properties including, but not limited to, certain carrier frequencies, pulse durations, pulse repetition frequencies, burst durations, and/or power levels, the neurons in that region of the brain may become more or less active (e.g., as measured by the rate at which they generate action potentials). Ultrasound transducers may be used to send focused ultrasound radiation through the skull and into one or more regions of the brain to selectively activate and/or inhibit groups of neurons. For example, in using ultrasound for neuromodulation, ultrasound radiation may be transmitted at the scalp through the entire thickness of the skull and through a certain distance of brain tissue (e.g., on the order of 10 cm or less, or another suitable distance).

Potential applications may include seizure suppression, chronic pain relief, neural function restoration, treatment of psychiatric disorders, etc. Conventional transducers can only focus ultrasound energy in one location of the brain. The steering capability is limited to a center region of the brain, and as such the scope of conventional transducers to address neurological conditions is limited. This means that each patient may require a bespoke transducer and as the treatment evolves, the caregiver may need to replace the transducer, increasing the time and economic costs on both sides. Conventional devices may also require the control of all transducer elements (e.g., over 1024), which implies an increased level of complexity for the supporting electronics, hardware, and software, leading to an immense increase in cost and market price of the device.

To address the shortcomings in the current state of the art, the inventors have developed annular array transducers that provide electronic focusing to a range of on-axis or off-axis locations, termed herein “treatment envelope.” In other words, even one such transducer can focus the ultrasound radiation at different depths in the brain. By physically moving the device over the head, one can address different points in the brain. This provides a simple and elegant way to address all regions in the brain.

The inventors have appreciated that non-invasive neuromodulation may be central to treating diseases like stroke, multiple sclerosis, neuropathic pain, migraine, depression, etc. Some conventional treatments may utilize Transcranial Magnetic Stimulation (TMS), however, with poor spatial selectivity and penetration depth. Ultrasound neuromodulation is a competing technique with superior spatial selectivity and penetration depth, and potentially a wider spectrum of applications. Further, such techniques may provide for treatment that allows one or more transducers to be placed on the scalp of the person. Therefore the treatment may be non-invasive because no surgery is required to dispose the transducers on the scalp for delivering ultrasound radiation to one or more regions of the brain of the person.

In some aspects, the inventors have developed a device wearable by or attached to a person and including at least one annular array transducer configured to provide ultrasound radiation (or ultrasound energy or waves) to at least one region of the brain, e.g., to perform non-invasive neuromodulation or neurostimulation. In some embodiments, the device has a compact and flat form-factor, which can be patchable, wearable, or handheld. It may either be tethered to or untethered from a main controller or monitoring computer. The device may be miniaturized and fabricated on flexible circuit boards that can conform to a human head curvature and geometry. In some embodiments, the device may be integrated with an application-specific integrated circuit (ASIC) and electronics on a single chip.

### Design of Annular Array Transducer

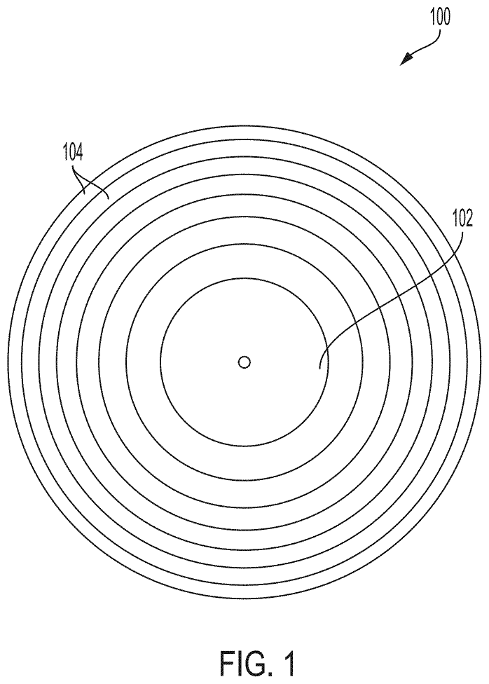

In some aspects, to achieve ultrasound focusing, the effective area (e.g., aperture) of the annular array transducer is subdivided into concentric elements having equal areas or substantially the same surface area (also referred to as channels herein). For example, FIG. 1 shows a front view of such an annular array transducer 100. In particular, FIG. 1 shows an 8-channel annular array transducer where the transducer material 102 (such as piezoelectric or another suitable material) is subdivided into concentric elements 104 using a dicing scheme represented by the black contour lines. Each element 104 of the annular array transducer 100 may be driven by electrical energy independently. The time-profile of the electrical energy can be in the form of continuous wave (CW), quasi CW, short pulse, or another suitable time-profile depending on the application. Each element may be assigned an independent phase or time-delay with respect to the other elements. This technique allows for electronic focusing on-axis with a large treatment envelope from a device with a flat form-factor.

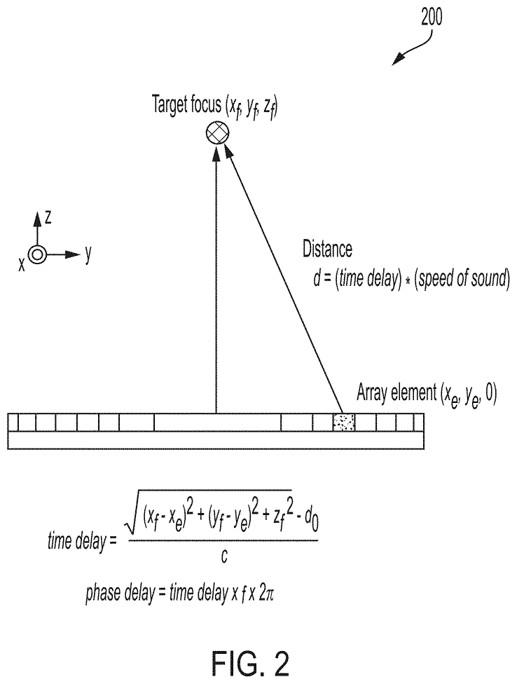

The number of channels may be any positive integer, with more channels resulting in better performance of the transducer. However, the increasing number of channels may pose difficulties in the hardware design and therefore may need to be limited in some embodiments. The inventors have found an 8-to 16-channel transducer to be viable, providing a proper balance between performance and hardware complexity, but the devices and techniques described herein are not so limited. The device may delay the signal of each channel based on the distance from the channel to a target focal depth in a region of the brain to ensure the resultant interference of the signals produces the target focusing. The calculations of time and phase delay for each element of an annular array transducer 200 are shown in FIG. 2. For an element with coordinates (xₑ, yₑ, 0) and a target focus (or target focal depth) with coordinates (x_{f}, y_{f}, z_{f}), the time delay and phase delay for the particular element may be calculated as follows:

$$time delay = \frac{\sqrt{{( {x}_{f} - {x}_{e} )}^{2} + {( {y}_{f} - {y}_{e} )}^{2} + {z}_{f}^{2}} - {d}_{0}}{c}$$

$$phase delay = time delay * f * 2 π$$

where:

d₀ is a distance between the target focus and a center element of the annular array transducer,

c is the speed of sound in tissue, e.g., 1486 m/s, 1500 m/s, or another suitable value, and

f is a center frequency of the annular array transducer.

In some embodiments, the device includes circuitry configured to assign a phase to each concentric element of the annular array transducer. The phase assigned to each concentric element may be independent from phases for other concentric elements. The phase assigned to each concentric element may be based on a distance from the concentric element to a target focal depth in a region of the brain. The target focal depth may be adjustable by adjusting the phase of one or more concentric elements. The range for adjustment of the target focal depth may depend on an aperture size of the device. For example, the target focal depth may be adjustable from F#0.5 to F#2.5 or another suitable range, where F# is a non-dimensional focusing metric defined as focal depth/full aperture.

### Considerations for Design of Individual Elements

In some embodiments, an annular array transducer having equal area elements may be able to keep a uniform ultrasound power across the elements. In some embodiments, one variant to the equal area embodiment may be elements with equal widths or substantially the same width. Equal width elements may be advantageous in certain configurations as the outer elements may provide less output power, which may naturally apodize the ultrasound radiation (or ultrasound beam). Apodization may be defined as amplitude weighting of the normal velocity across the aperture. In a single transducer, apodization can be achieved in many ways, such as by tapering the electric field along the aperture, by attenuating the beam on the face of the aperture, by changing the physical structure or geometry, or by altering the phase in different regions of the aperture. In arrays, apodization is accomplished by simply exciting individual elements in the array with different voltage amplitudes. One of the main reasons for apodization is to lower the “sidelobes” on either side of the main beam. Just as time sidelobes in a pulse can appear to be false echoes, strong reflectors in a beam profile sidelobe region can interfere with the interpretation of on-axis targets.

In some embodiments, there may be certain design considerations with regard to the geometric dimensions of each individual element. The piezoelectric material may have mechanical resonances in some or all three spatial dimensions. In the design of the annular array described herein, it may be desired to have only a thickness-mode resonance (e.g., piston-like movement of each element up and down). However, because of elasticity of each piezoelectric element, there may be lateral modes that can interfere with the main thickness-mode resonance and create spurious field patterns and defocusing. This problem may be mitigated via one or more of the methods described below.

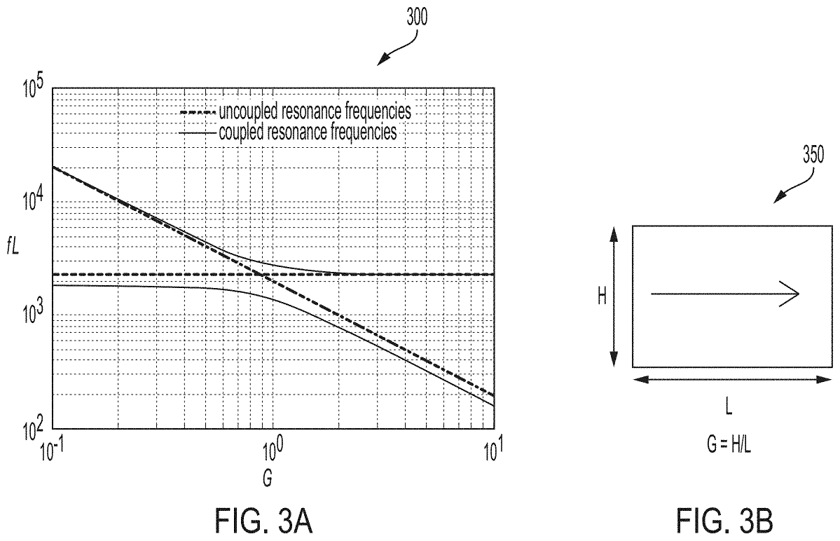

In some embodiments, the segmenting scheme and aperture may be chosen in a range in which the lateral modes are uncoupled from the main thickness-mode resonance. FIGS. 3A-3B (300, 350) show the behavior of longitudinal and lateral modes as a function of frequency and aspect ratio. For an optimal design, the aspect ratio may be chosen such that the different resonances have a large separation.

In some embodiments, for elements larger than the desired aspect ratio, the elements may be sub-diced, however, keeping them electronically in-phase (e.g., shorted).

In some embodiments, a transducer material may be chosen with a minimal lateral mode-coupling (e.g., 1-3 composites, lead metaniobate, single-crystal piezoelectric materials, composite piezoelectric materials, or another suitable transducer material).

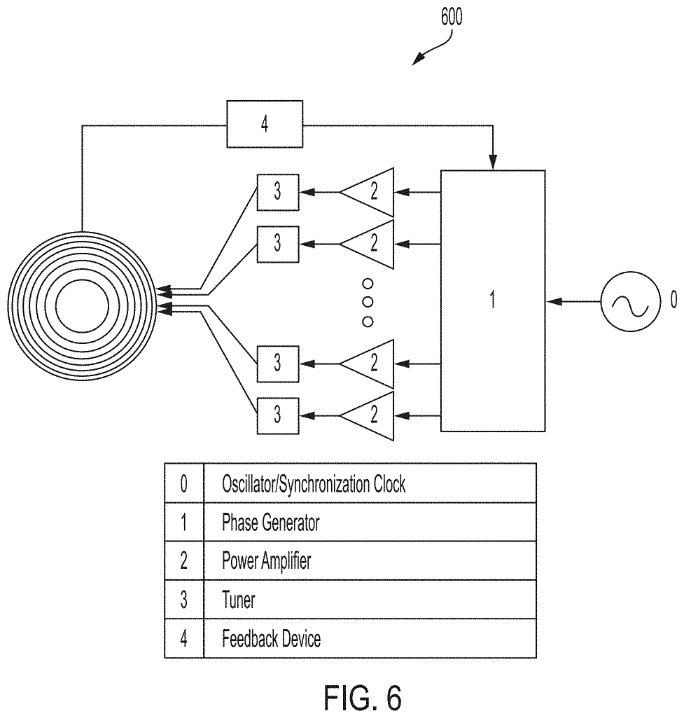

In some embodiments, phase correction may be provided to each individual element using a feedback loop. The feedback metric can be the impulse response of the transducer or electrical characteristics such as the impedance, admittance, or RF reflection metrics such as “S” parameters. In some embodiments, each transducer element may be electrically tuned to suppress spurious resonances. More details on the feedback loop are provided further below with respect to FIG. 6.

### Fabrication of Annular Array Transducer

In some aspects, the transducer may be fabricated by radially dicing a piezoelectric disc into a number of concentric elements, or channels, as illustrated in FIG. 1. Fabrication techniques, such as computer numerical control (CNC) machining, ultrasonic cutting, diamond core-drilling, ultrasonic drilling, laser cutting, water-jet cutting, or another suitable technique, may be utilized to dice a transducer raw material such as a piezoelectric disc into a desired annular shape. The first step in the fabrication process may be to deposit thin metal layers on the desired faces of the transducer material (such as top and bottom faces of a piezoelectric disc). The metal layers (also known as electrodes) provide electrical connections to the transducer material. After that the transducer material is poled under a high electric field. Poling creates the piezoelectric property in the raw material by making it sensitive to deformation along the poled direction. Next, a thin layer of polymer, known as the matching layer, is cast on the outside face of the material. The purpose of the matching layer is to match the acoustic impedance of the transducer to tissue or any lower impedance medium.

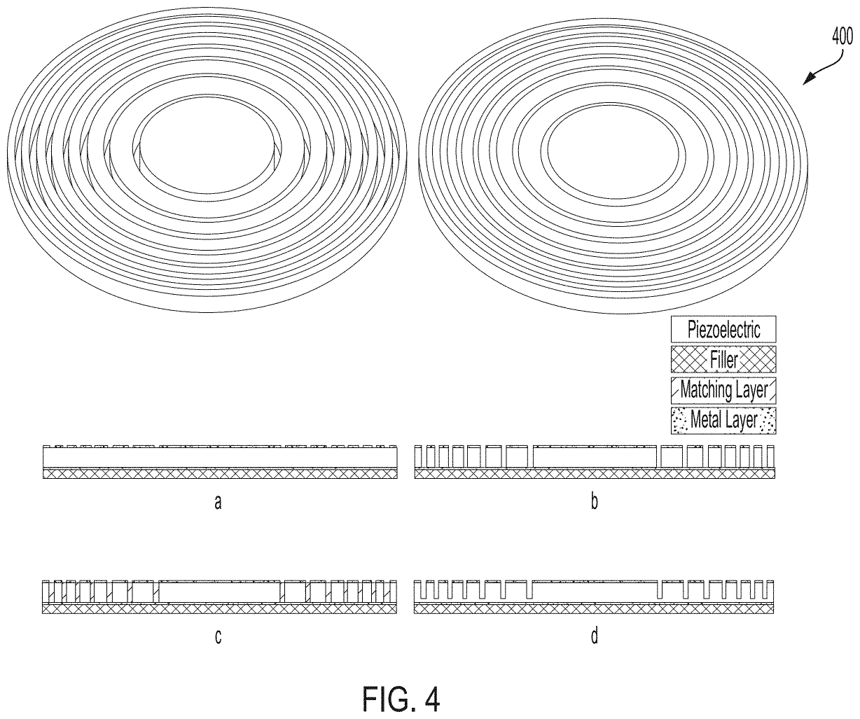

An annular build may be achieved via several approaches 400 as shown in FIG. 4. In one approach, only the electrodes may be patterned into an annular (multi-concentric-ring) pattern (FIG. 4, a). A mask may be used to define the electrode pattern. The mask exposes the areas that need to be removed and covers the electrode areas. Next, the metal layers are etched away using one or more suitable etching techniques.

The second approach is to dice all the way into the transducer material (FIG. 4, b). This approach may provide excellent decoupling of the mechanical vibration of each element (e.g., to help reduce crosstalk). This approach can be further optimized, if necessary, by filling the spacings between the elements with polymers such as epoxy (FIG. 4, c). This may help transducer elements primarily resonate in the thickness mode.

An alternative approach for dicing the elements may be to dice partially into the transducer material (FIG. 4, d). This approach may provide a simpler fabrication, however, at the cost of more lateral coupling and crosstalk between the neighboring elements.

In the above-explained approaches, the lateral spacing between the neighboring elements may be minimized to preserve as much transducer area as possible. The spacing may be limited by the dicing technique such as size of the CNC mill bits.

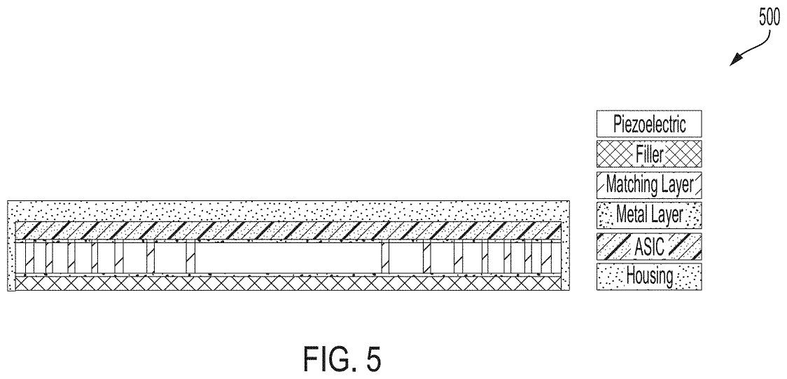

In some embodiments, the annular array may be bonded onto a circuit board which hosts an ASIC (or ultrasound chip) and provides electrical connections to a computer or any outside device. FIG. 5 shows an exemplary diagram of an annular array transducer 500 integrated with an ASIC and housing.

### Electronics of Annular Array Transducer

In some embodiments, the electrical connections to each individual element can be realized via soldering or bonding wires directly onto each element. The interface facing outside (e.g., patient) serves as the common ground. The other end of the end wires can connect to the ultrasound chip providing adequate power and phase control to drive the elements. FIG. 6 shows an exemplary block diagram of the main components of the ultrasound chip 600. In some embodiments, piezoelectric elements can be directly bonded to the ASIC/chip on a printed circuit board (PCB). This may be advantageous as it minimizes electronic noise. The PCB may provide a supporting or backing material for the transducer which may help suppress spurious modes.

In FIG. 6, the components shown include an oscillator (0), a phase generator (1) coupled to the oscillator, power amplifiers (2) coupled to the phase generator, tuners (3) coupled to the power amplifiers, an annular array transducer coupled to the tuners, and feedback circuitry (4) coupled to the annular array transducer and the phase generator. To implement this system, a master clock may be used for synchronization among phase generators (e.g., digital synthesizers). The number of phase generators may match the number of transducer elements. The clock circuitry can also generate the master AC waveform which is then fed into the phase generator circuitry. The phase generator delays each waveform according to the desired phase for each transducer element. The delayed waveforms are then amplified and fed into a tuning network. The tuning network matches the impedance and bandwidth of the input signals to the desired values that are fed into each transducer element.

In some embodiments, the system provides a feedback loop by monitoring a performance metric (e.g., impedance, admittance, S parameter, impulse response, etc.) of each transducer element. This feedback loop may be used to optimize the performance by modifying the amplitude, phase, and/or frequency of the waveform of each channel. The input waveform can be set as a CW, quasi CW (e.g., a long burst), or a short pulse. For example, the feedback device or circuitry may receive echo data from the annular array transducer and transmit the echo data to the phase generator. For example, the echo data may include echoes returning from the tissue and received by the same annular array transducer. The phase generator may correct an amplitude and/or a phase of the ultrasound radiation based on the echo data.

In some embodiments, the device can act as a standalone feedback mechanism without being dependent on a secondary guiding system. In this embodiment, the annular array elements transmit a focused on-axis quasi-CW pulse by applying nominal phases (e.g., time delays). The echoes returning from the tissue are received by the same annular elements and focused via the delay-and-sum beamforming process or similar processing techniques. The signals are then demodulated to obtain the phase and envelope information of the echoes. The beams may be constantly monitored to match certain criteria such as the maximum amplitude or energy of the received signal.

In some embodiments, the device may act in dual-mode by switching back and forth between the quasi-CW and pulsed mode. In the pulsed mode, the device can monitor the effect of quasi-CW mode such as tissue displacement by monitoring the phase of the beam or similar metrics.

In some embodiments, the device can be equipped with imaging capability for guidance. This may be achieved via conventional ultrasonography where the ultrasound images are acquired in reflection, or pulse-echo mode. When imaging, the annular array elements transmit a focused pulse by applying the correct time delay. The echoes returning from the tissue are received by a secondary collocated device and focused via the delay-and-sum beamforming process or similar processing techniques. The signals are then demodulated to obtain the phase and envelope information of the echoes.

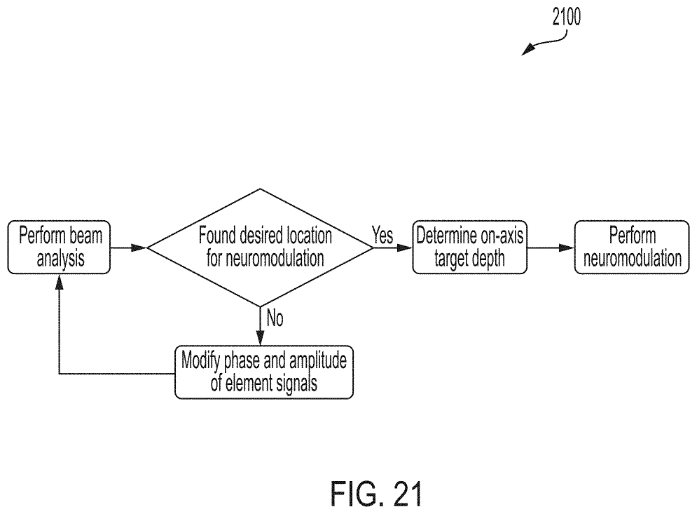

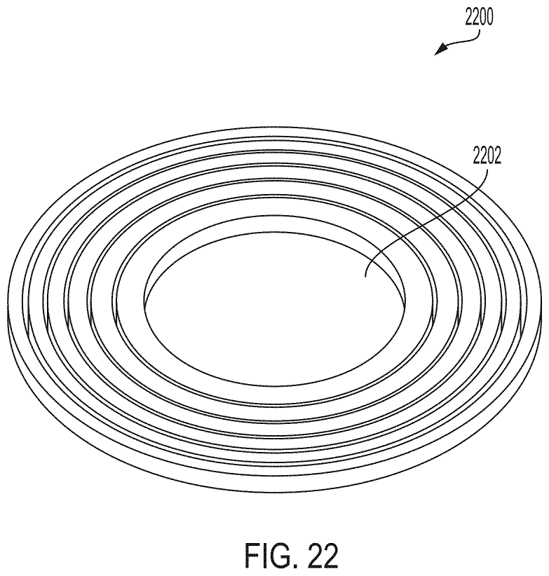

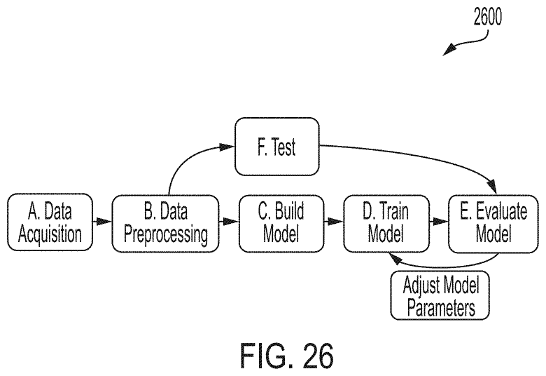

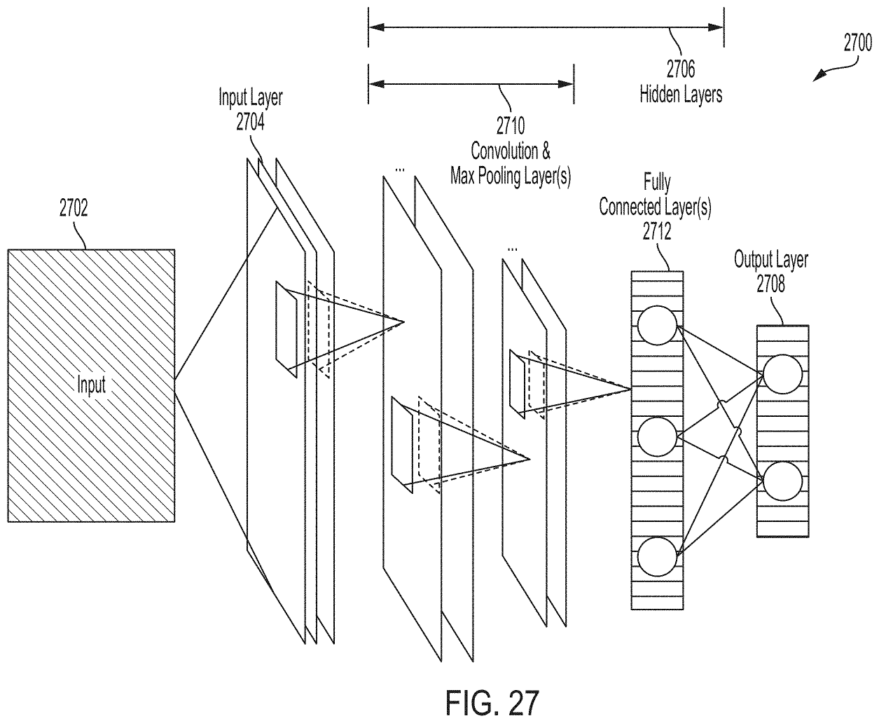

The received beams/images can be further processed through machine learning algorithms (e.g., as described with respect to FIG. 26 or FIG. 27 or another suitable machine learning algorithm) to identify the exact target location for delivering ultrasound radiation for neuromodulation, after which the transducer is fixed at this desired on-head location and the ultrasound beam is re-focused at the target with higher intensity for neuromodulation. Apodization might be needed in certain cases, but because of the equal area of the elements, the apodization may naturally occur in most cases. FIG. 21 shows an exemplary workflow 2100 for a neuromodulation device equipped with guiding capabilities, including performing beam analysis, finding desired location for neuromodulation (and if not, modifying phase and amplitude of element signals and trying again), determining on-axis target focal depth, and performing neuromodulation or another suitable non-invasive ultrasound radiation based therapy.

In some embodiments, the devices and techniques described herein can be equipped with a wearable and stereotactic robotic head-mount with the capability of moving the probe or fixing it to an exact location.

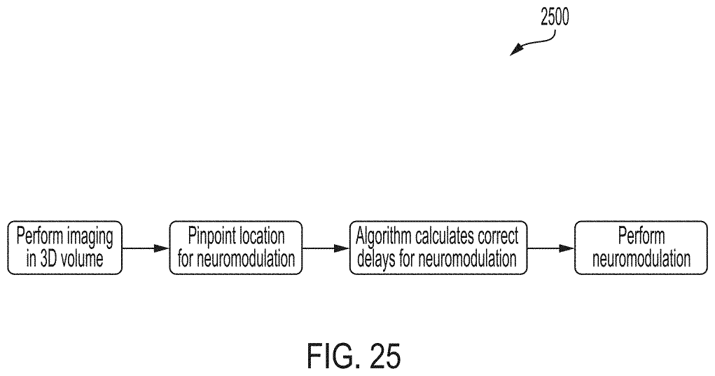

In some embodiments, 3D scanning capability may improve the workflow of neuromodulation by guiding the beam in 3D. FIG. 25 shows an exemplary workflow 2500 for a neuromodulation device equipped with 3D/volumetric scanning capabilities, including performing imaging in 3D volume, finding location for neuromodulation, calculating correct delays for neuromodulation, and performing neuromodulation.

### Guidance and Beam Navigation

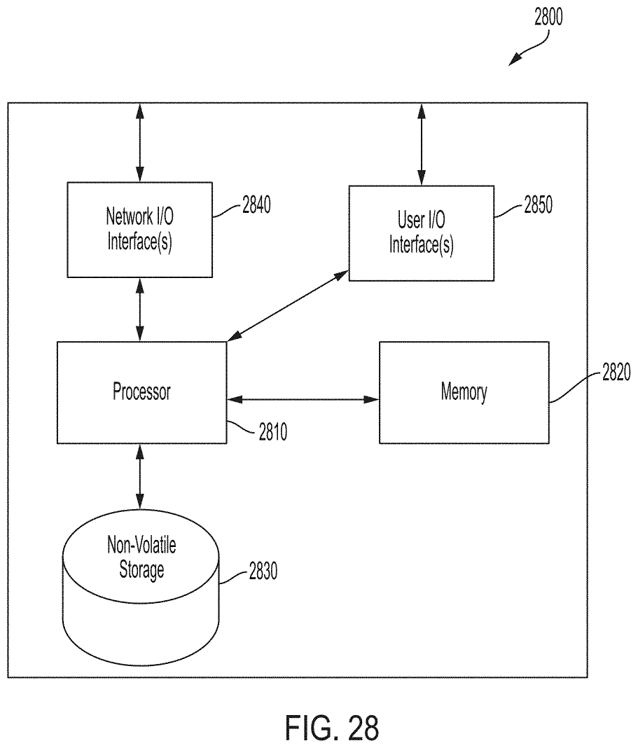

In some embodiments, the device described herein can be combined with any method of guidance for navigating the ultrasound radiation or beam to a region of the brain including, but not limited to, ultrasound, optoacoustics, photoacoustics, thermo-acoustics, doppler and functional ultrasound, magnetic resonance-based radiation force imaging (RFI), shear wave elastography, magnetic resonance thermometry, functional imaging techniques, electroencephalography, optical tracking, and/or simulation-based guidance. The device can combined with the above mentioned modalities for a responsive neurostimulation (RNS) device. In some embodiments, the device includes a processor (e.g., a processor described with respect to FIG. 28) to guide the ultrasound radiation to a region of the brain using portable magnetic resonance (MR) imaging, including high-field, mid-field, low-field, very low-field, and/or ultra-low field MR imaging. For example, the portable MR imaging may be provided by a HYPERFINE SWOOP portable MRI machine. In some embodiments, such a portable MRI machine may be moved to a patient's bedside as needed and MR images may be acquired within a short period of time, e.g., on the order of minutes, or another suitable time period. The device may receive the acquired MR images and process them to guide the ultrasound radiation to one or more suitable regions of the brain.

As used herein, “high-field” refers generally to MRI systems presently in use in a clinical setting and, more particularly, to MRI systems operating with a main magnetic field (i.e., a BO field) at or above 1.5 T, though clinical systems operating between 0.5 T and 1.5 T are often also characterized as “high-field.” Field strengths between approximately 0.2 T and 0.5 T have been characterized as “mid-field” and, as field strengths in the high-field regime have continued to increase, field strengths in the range between 0.5 T and 1 T have also been characterized as mid-field. By contrast, “low-field” refers generally to MRI systems operating with a BO field of less than or equal to approximately 0.2 T, though systems having a BO field of between 0.2 T and approximately 0.3 T have sometimes been characterized as low-field as a consequence of increased field strengths at the high end of the high-field regime. Within the low-field regime, low-field MRI systems operating with a BO field of less than 0.1 T are referred to as “very low-field” and low-field MRI systems operating with a BO field of less than 10 mT are referred to as “ultra-low field.”

### Transducer Technology

Transducers can be of a variety of types such as piezoelectric transducers, capacitive micromachined ultrasonic transducers (CMUTs), piezoelectric micromachined ultrasonic transducer (PMUTs), electromagnetic acoustic transducers (EMATs), and other suitable transducers. Material and dimensions may determine the bandwidth and sensitivity of the transducer. While the devices and techniques described herein are described with respect to piezoelectric technology, these devices and techniques may be equally applicable to other types of transducer technology. For example, CMUTs may be of particular interest as they can be easily miniaturized even at low frequencies, have superior sensitivity as well as wide bandwidth.

### Exemplary Annular Array Embodiments

In some aspects, the inventors have developed annular array devices that have maximal transmit power at around 400-500 kHz and 800-1000 kHz, with respectively two- and three-inch apertures, but the devices and techniques described herein are not so limited. For example, the described annular array devices may have maximal transmit power at a center frequency between 200 kHz to 1000 kHz, or another suitable range. In some embodiments, the center frequency may depend on a backing material for the device. In another example, the described annular array devices may have an aperture size between one inch and four inches, or another suitable range. In yet another example, the annular array device may have a fractional bandwidth range from 10% to 60% of a center frequency for the device, or another suitable range. The inventors have developed 8-channel and 16-channel phasing schemes that can sweep the focus in a wide range of depths (e.g., from F#0.5 to F#2.5, or another suitable range; F# is a non-dimensional focusing metric defined as focal depth/full aperture). In some embodiments, finite element simulations may be used to validate the results.

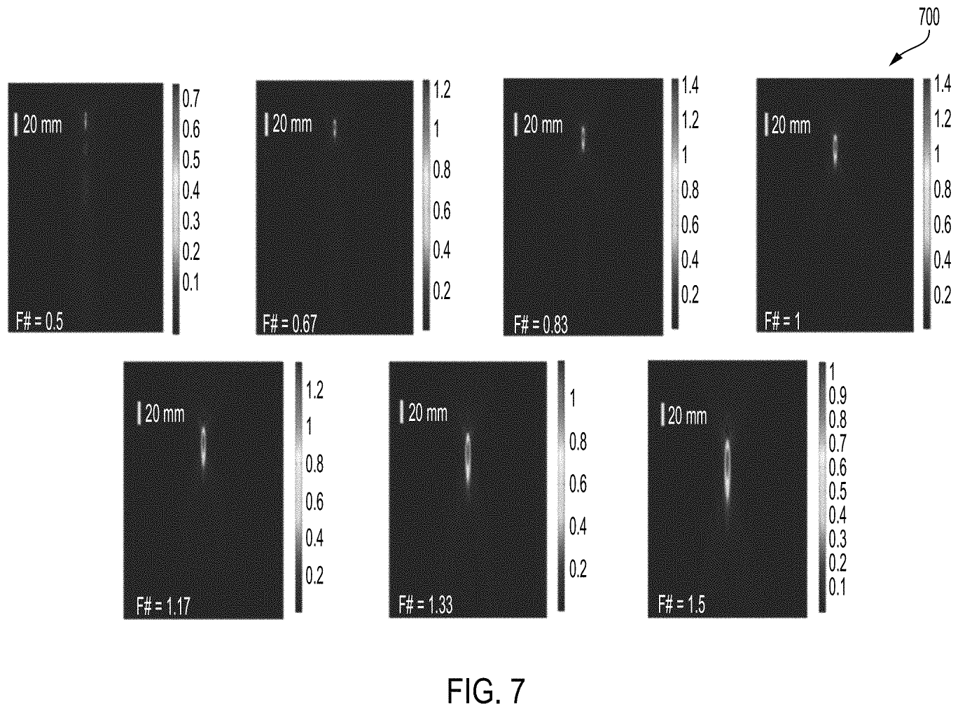

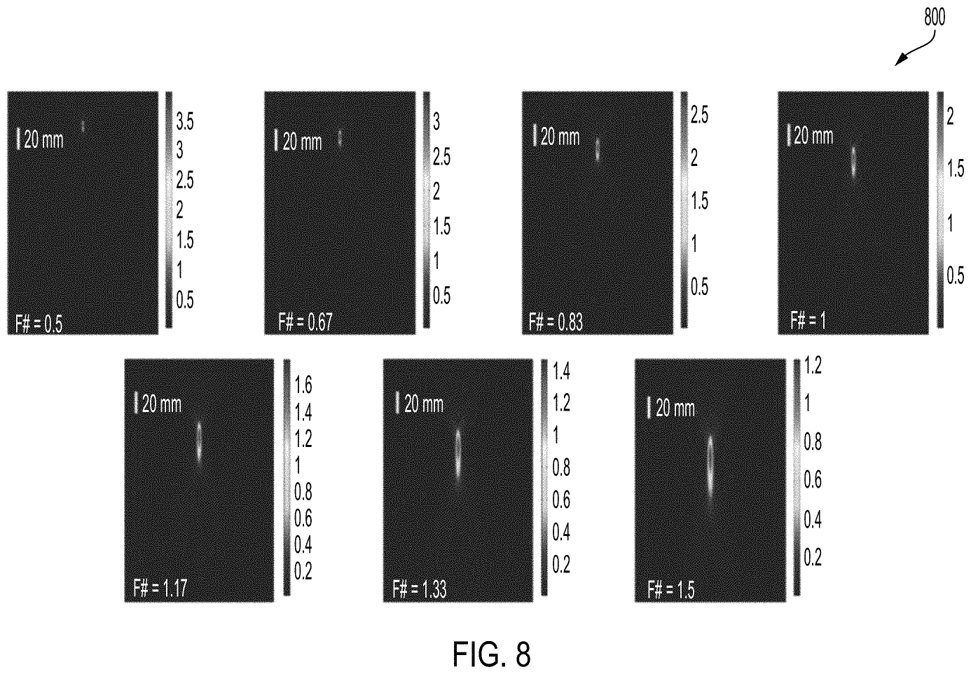

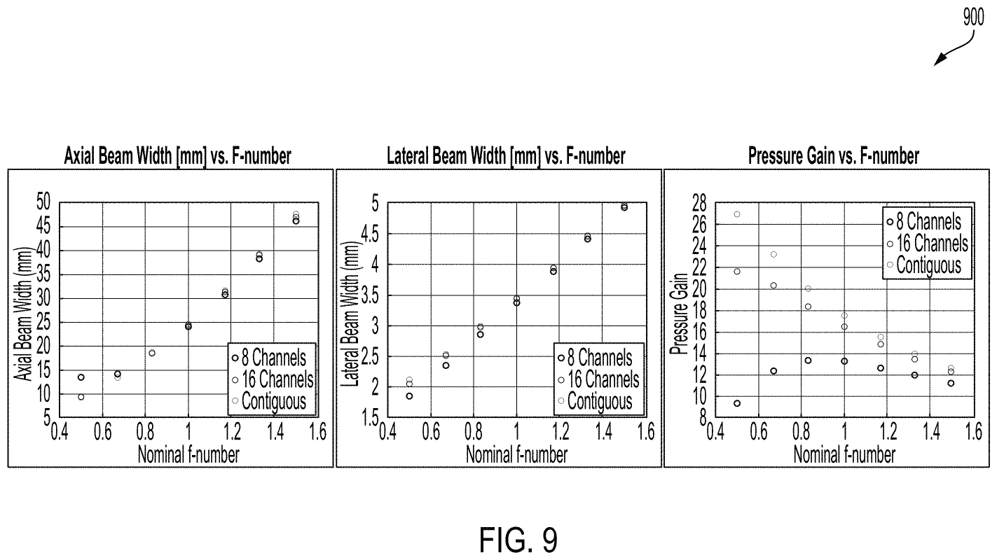

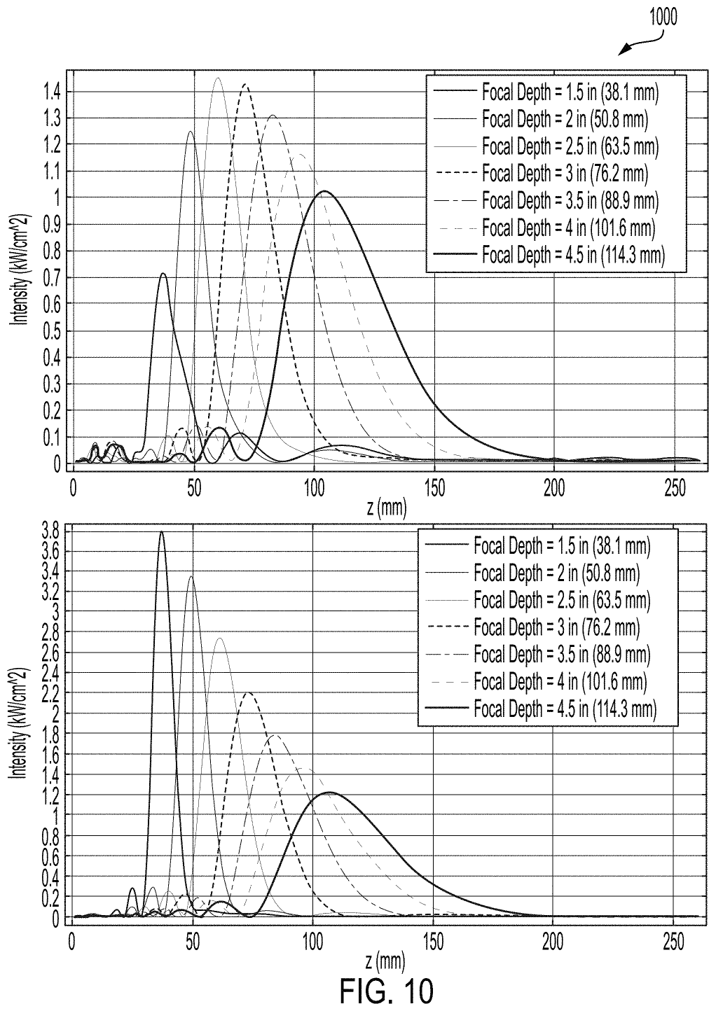

With respect to the 3-inch aperture and 500 kHz design, FIGS. 7-10 illustrate the focusing performance (e.g., intensity plots) for a 500 kHz transducer with a 3-inch aperture, for a 0.5 MPa input surface pressure on each individual element. The transducer is located at the top boundary. FIG. 7 shows illustrative plots 700 for focusing performance for an 8-channel design with a 3-inch aperture at 500 kHz, as a function of F#. The measurements shown are in kW/cm². FIG. 8 shows illustrative plots 800 for focusing performance for a 16-channel design with a 3-inch aperture at 500 kHz, as a function of F#. The measurements shown are in kW/cm². FIG. 9 shows illustrative plots 900 for performance comparisons between the 8-channel, 16-channel, and ideal (e.g., contiguous design) cases, with a 3-inch aperture at 500 kHz. FIG. 10 shows illustrative plots 1000 for performance comparisons between the 8-channel (top) and 16-channel (bottom), with a 3-inch aperture at 500 kHz.

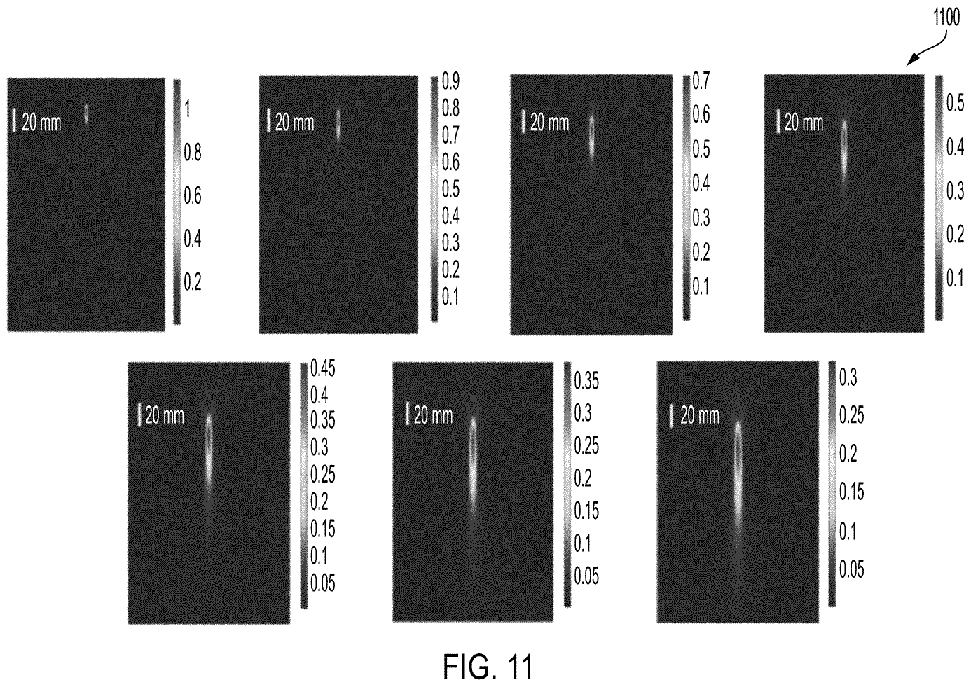

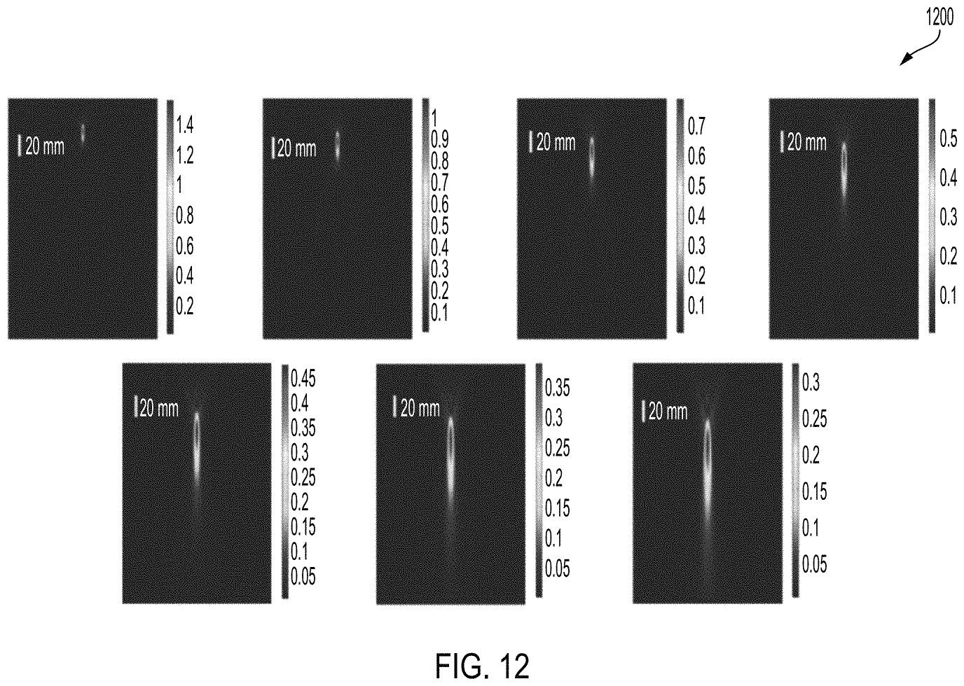

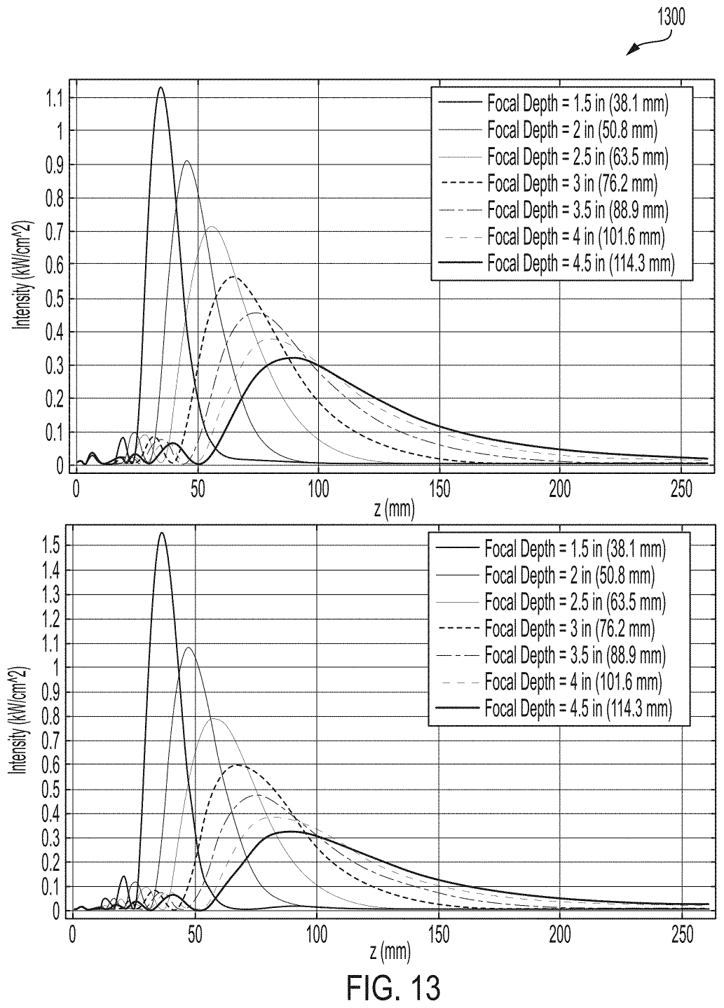

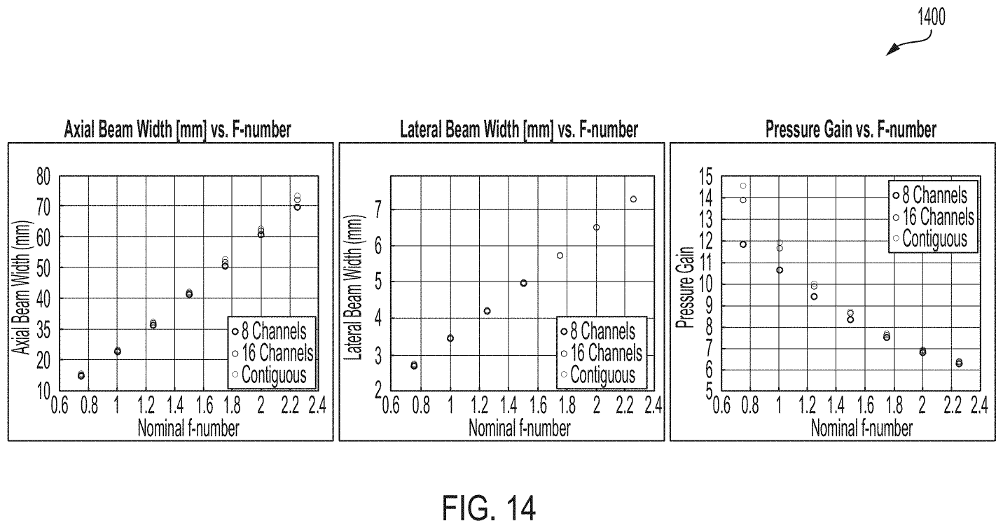

With respect to the 2-inch aperture and 500 kHz design, FIGS. 11-14 illustrate the focusing performance (e.g., intensity plots) for a 500 kHz transducer with a 2-inch aperture, for a 0.5 MPa input surface pressure on each individual element. The transducer is located at the top boundary. FIG. 11 shows illustrative plots 1100 for focusing performance for an 8-channel design with a 2-inch aperture at 500 kHz, as a function of F#. The measurements shown are in kW/cm². FIG. 12 shows illustrative plots 1200 for focusing performance for a 16-channel design with a 2-inch aperture at 500 kHz, as a function of F#. The measurements shown are in kW/cm². FIG. 13 shows illustrative plots 1400 for performance comparisons between the 8-channel (top) and 16-channel (bottom), with a 2-inch aperture at 500 kHz. FIG. 14 shows illustrative plots 1300 for performance comparisons between the 8-channel, 16-channel, and ideal (e.g., contiguous design) cases, with a 2-inch aperture at 500 kHz.

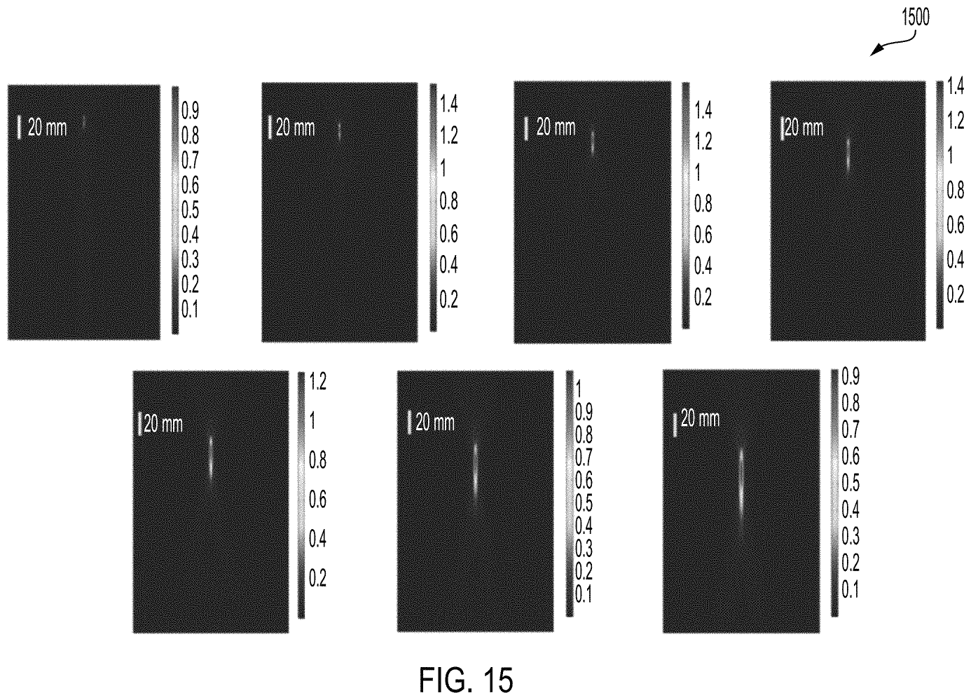

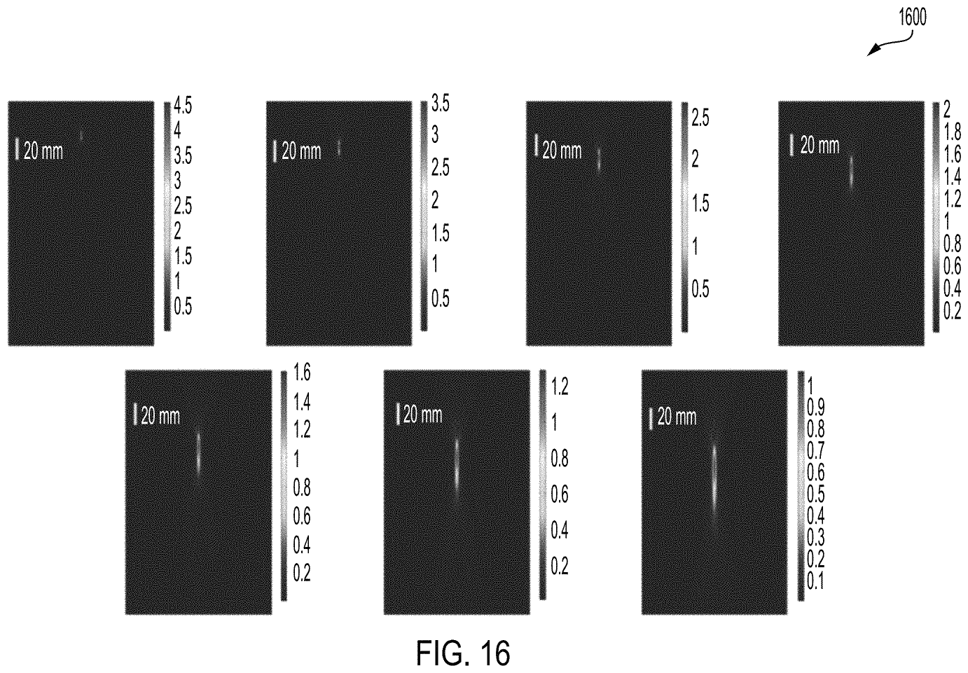

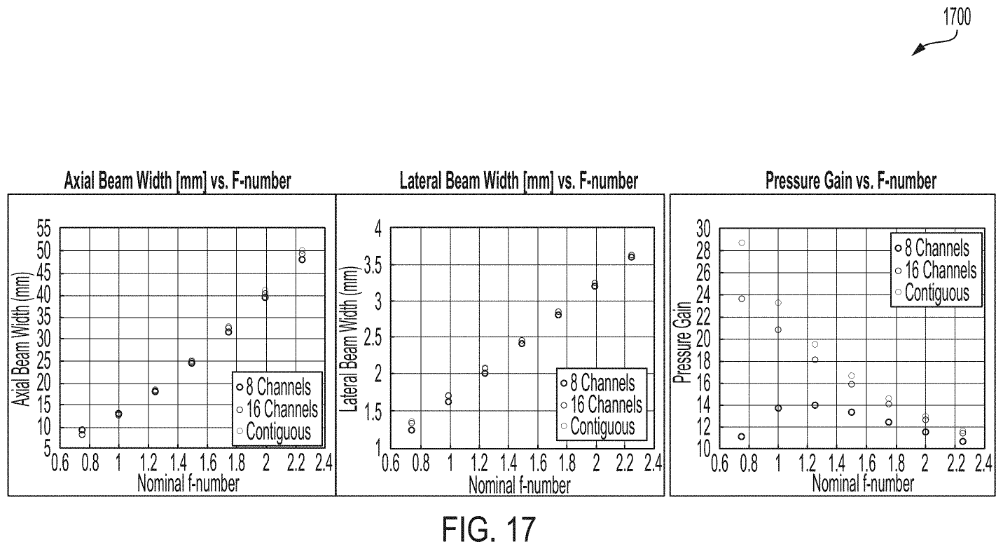

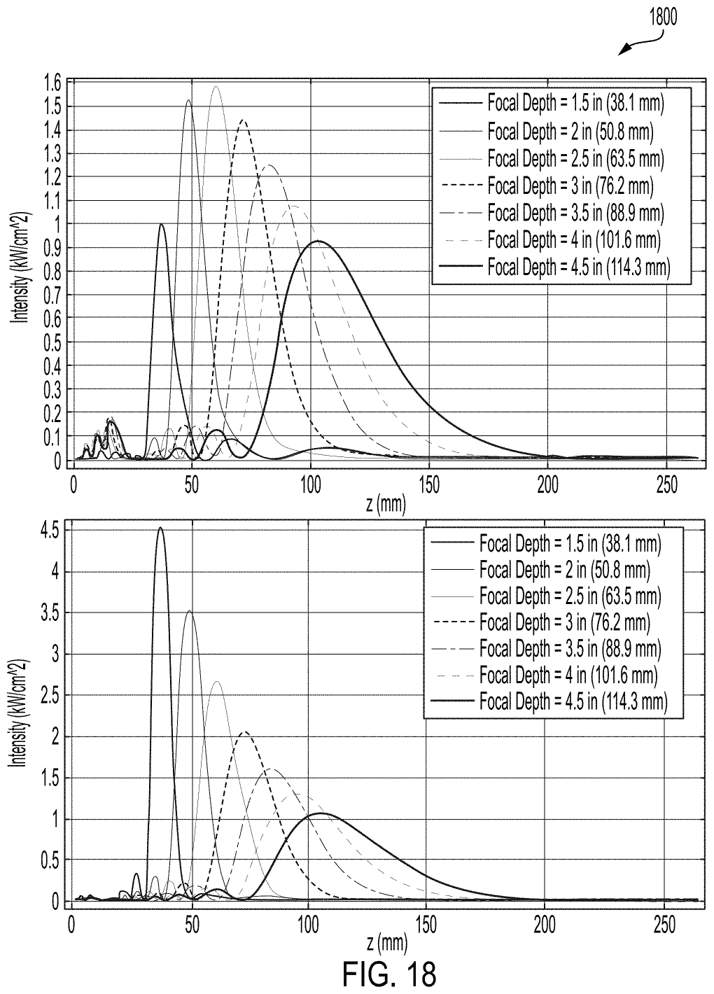

With respect to the 2-inch aperture and 1 MHz design, FIGS. 15-18 illustrate the focusing performance (e.g., intensity plots) for a 1 MHz transducer with a 2-inch aperture, for a 0.5 MPa input surface pressure on each individual element. The transducer is located at the top boundary. FIG. 15 shows illustrative plots 1500 for focusing performance for an 8-channel design with a 2-inch aperture at 1 MHz, as a function of F#. The measurements shown are in kW/cm². FIG. 16 shows illustrative plots 1600 for focusing performance for a 16-channel design with a 2-inch aperture at 1 MHz, as a function of F#. The measurements shown are in kW/cm². FIG. 17 shows illustrative plots 1700 for performance comparisons between the 8-channel, 16-channel, and ideal (e.g., contiguous design) cases, with a 2-inch aperture at 1 MHz. FIG. 18 shows illustrative plots 1800 for performance comparisons between the 8-channel (top) and 16-channel (bottom), with a 2-inch aperture at 1 MHz.

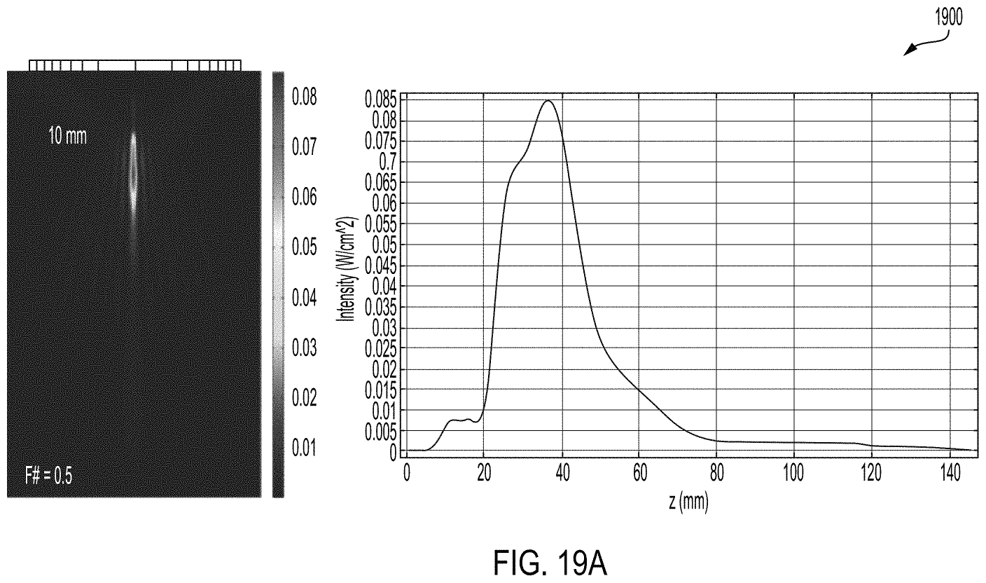

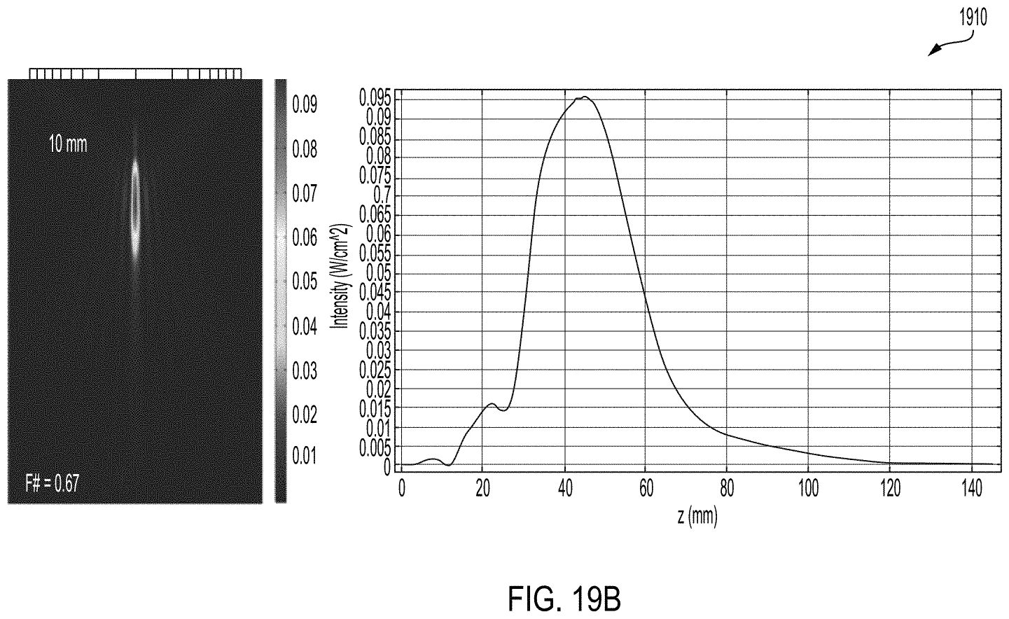

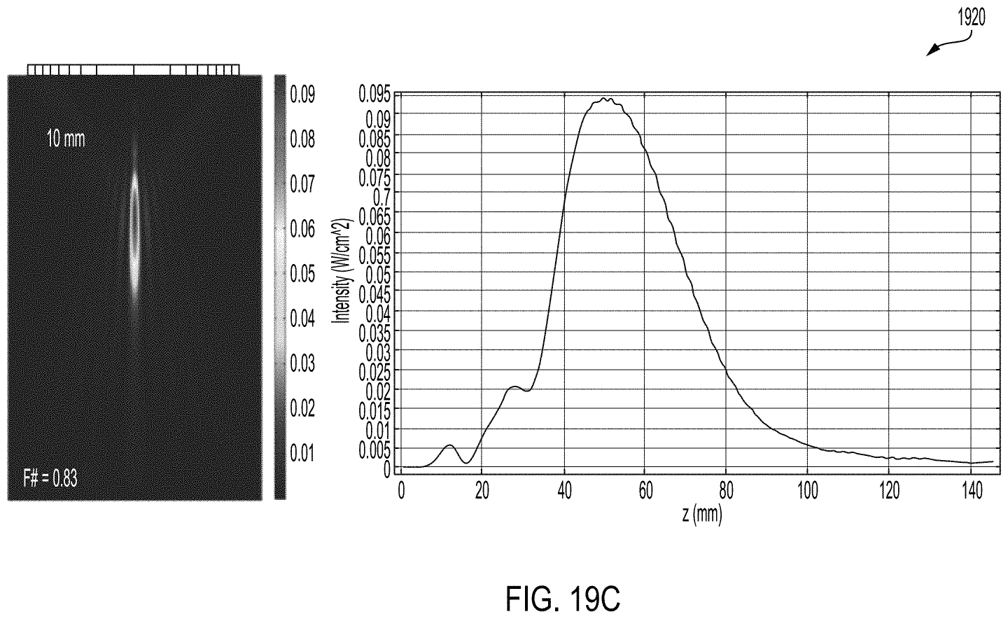

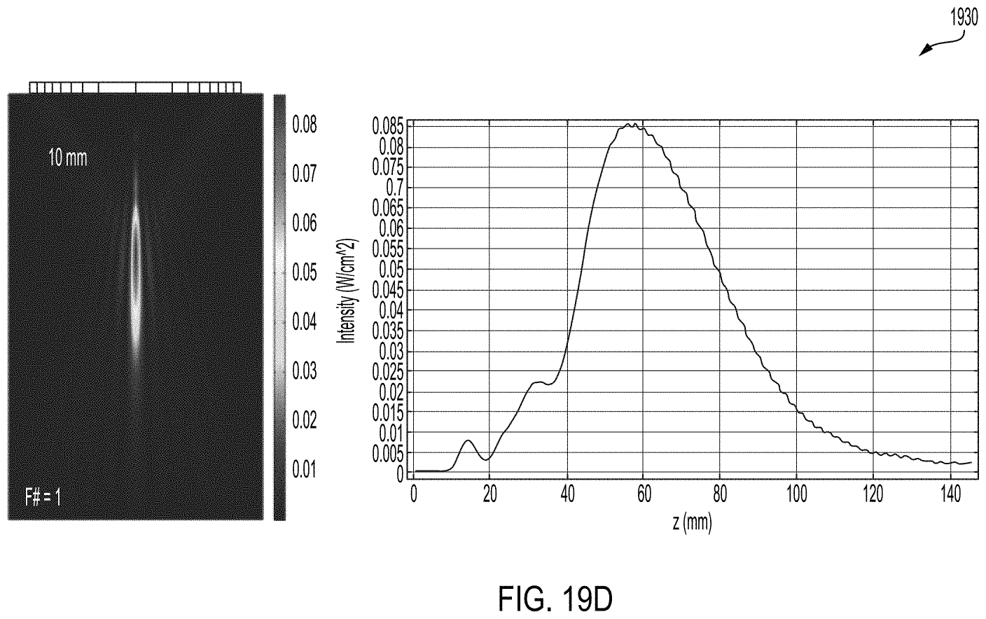

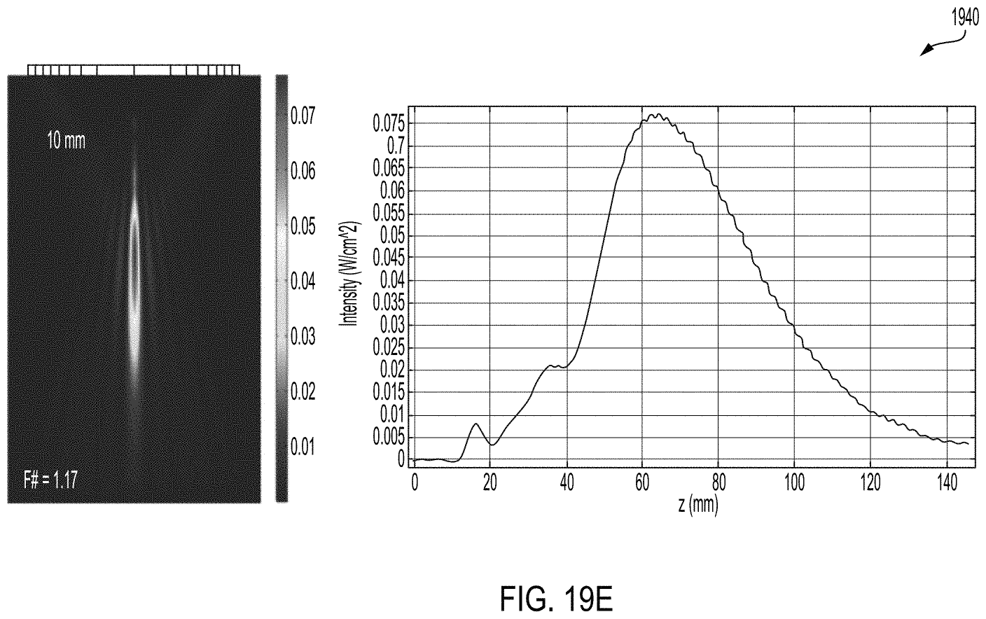

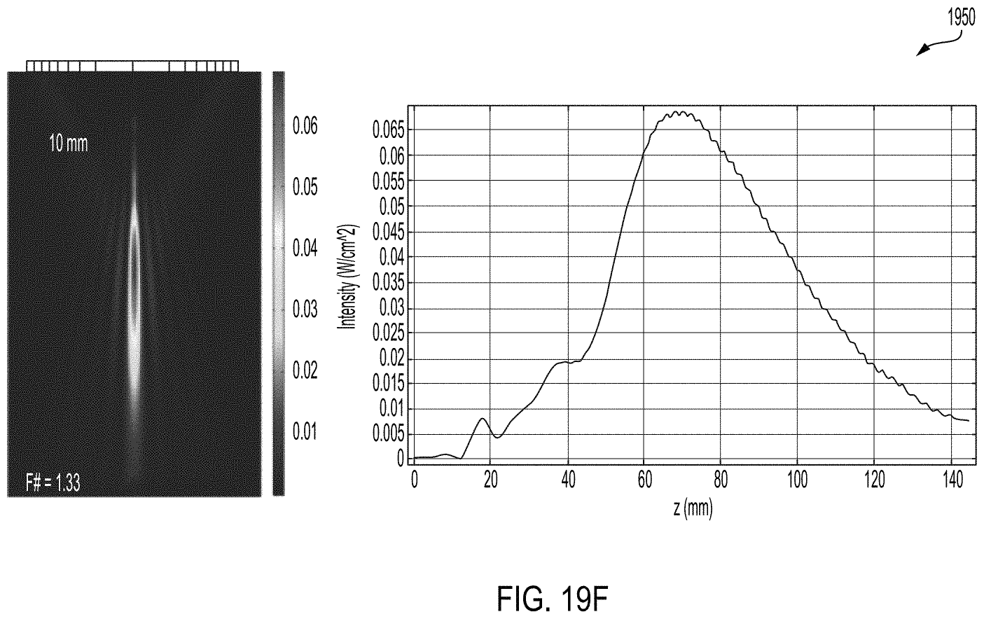

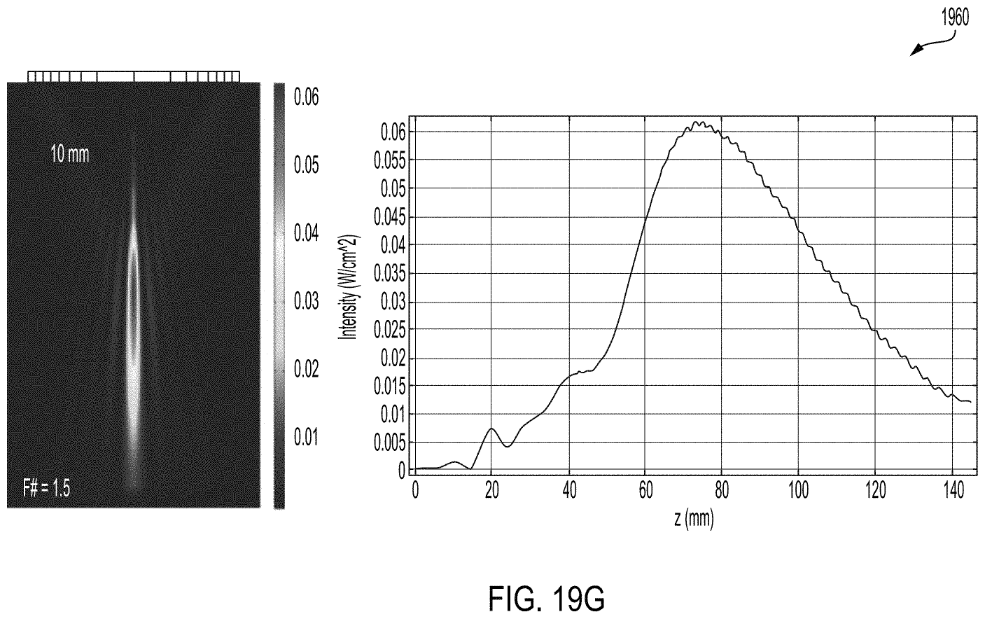

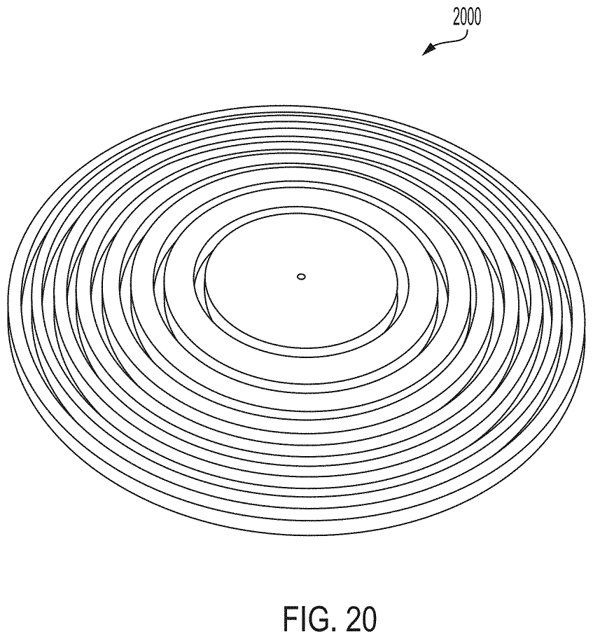

The inventors have also developed and validated performance of a piezoelectric annular array with an 8-channel, 3-inch aperture, and 400-500 kHz design. FIGS. 19A-19G show illustrative plots 1900, 1910, 1920, 1930, 1940, 1950, and 1960 for focusing performance for the 8-channel design with a 3-inch aperture at 500 kHz, as a function of F#. The measurements shown are in kW/cm².

### Exemplary Fabricated Annular Array

FIG. 20 shows an illustrative embodiment of a fabricated annular array transducer 2000. In particular, FIG. 20 shows an annular array transducer fabricated based on the configuration of FIG. 4, d. The device shown in FIG. 20 has been fabricated using a CNC machining technique. The device is an 800 kHz device with a 2-inch aperture. The piezoelectric disc is 2.5 mm. The trenches are 1 mm wide and 2.3 mm deep.

### Other Exemplary Configurations

In some aspects, in order to accommodate imaging for guiding the ultrasound radiation or beam, the annular array design may be modified as shown in FIG. 22. The modified design for annular array transducer 2200 includes an open middle area 2202 that allows for insertion of an imaging array in the middle of the annular array. This embodiment builds on top of the design in FIG. 20 where imaging and neuromodulation can be performed on the axis. While the modified design may give better manufacturability, the annular array has fewer elements in total, and as such has lower sensitivity and reduced range of on-axis imaging and output intensity.

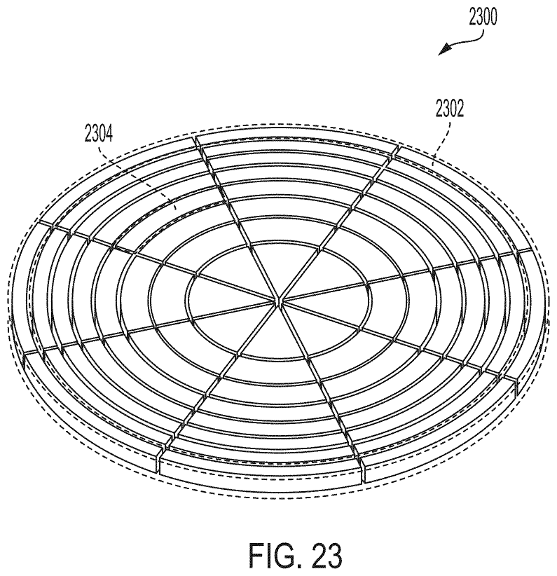

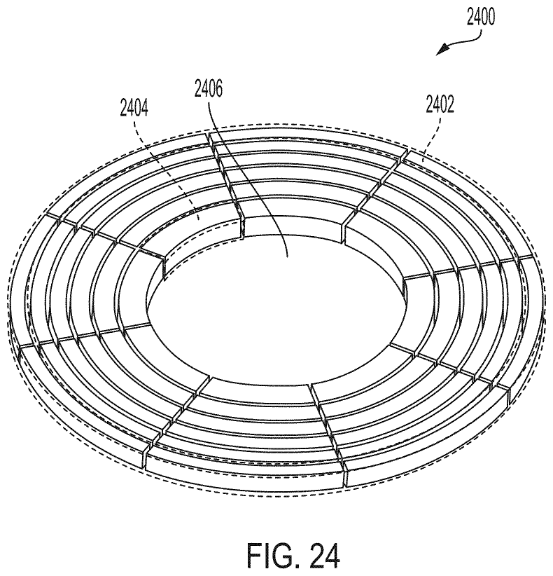

In some aspects, because an annular array with axial symmetry can only focus at on-axis points, the inventors have developed other designs that introduce beam-steering functionality to off-axis points by segmenting the array elements circumferentially as well as radially. This means the transducer can have off-axis scan lines and generate images in a three-dimensional (3D) volume. As a result, the transducer can have a larger field of view and image more than the axis in front. Both designs in FIG. 23 and FIG. 24 demonstrate the 3D imaging embodiment as they have elements distributed in two dimensions of the polar system. In particular, FIG. 23 and FIG. 24 illustrate annular array transducer designs 2300 and 2400 with radial and circumferential partitioning of the elements. Like the design in FIG. 22, the design illustrated in FIG. 24 includes an open middle area 2402 that allows for insertion of an imaging array in the middle of the annular array 2400.

FIG. 23 and FIG. 24 illustrate annular arrays 2300 and 2400 with segmentation along the circumference. For example, the annular array 2300 may be fabricated by radially dicing a piezoelectric material into concentric segments, such as concentric segment 2302, and circumferentially dicing each concentric segment into elements, such as element 2304. Each element may be assigned an independent phase, as opposed to an annular array segmented only radially where each annulus or concentric segment has only one phase. With circumferential segmentation the array may focus at any point in three dimensions, within the field of view of the array. In another example, the annular array 2400 may be fabricated by radially dicing a piezoelectric material into concentric segments, such as concentric segment 2402, and circumferentially dicing each concentric segment into elements, such as element 2404. Each element may be assigned an independent phase. Further, the annular array 2400 includes an open middle area 2406 that allows for insertion of an imaging array in the middle of the annular array 2400.

In some embodiments, a device includes an annular array transducer including concentric segments, such as concentric segment 2302 or 2402, and each concentric segment includes elements, such as element 2304 or 2404, along a circumference of the concentric segment. One or more of these elements may be operable to provide ultrasound radiation. The device may include circuitry configured to independently drive electrical energy to each element, such as circuitry shown and described with respect to FIG. 6. For example, a time profile of the electrical energy includes a continuous wave, a quasi-continuous wave, and/or a pulsed wave. The circuitry may be used to assign a phase to each element independent from phases for other elements. The phase assigned to each element may be based on a distance from the element to a target focal depth in a region of the brain. The target focal depth may be adjustable in up to three dimensions by adjusting the phase of one or more elements.

### Exemplary Machine Learning Architecture

FIG. 26 shows workflow 2600 for steps that may be undertaken to construct and deploy the algorithms described herein, including data acquisition, data preprocessing, building a model, training the model, evaluating the model, testing, and adjusting model parameters.

FIG. 27 shows a convolutional neural network 2700 that may be employed by the devices and techniques described herein. The statistical or machine learning model described herein may include the convolutional neural network 2700, and additionally or alternatively another type of network, suitable for predicting frequency, amplitude, acoustic beam profile, and other requirements, such as expected temperature elevation and/or radiation force, etc. As shown, the convolutional neural network comprises an input layer 2704 configured to receive information about the input 2702 (e.g., a tensor), an output layer 2708 configured to provide the output (e.g., classifications in an n-dimensional representation space), and a plurality of hidden layers 2706 connected between the input layer 2704 and the output layer 2708. The plurality of hidden layers 2706 include convolution and pooling layers 2710 and fully connected layers 2712.

The input layer 2704 may be followed by one or more convolution and pooling layers 2710. A convolutional layer may comprise a set of filters that are spatially smaller (e.g., have a smaller width and/or height) than the input to the convolutional layer (e.g., the input 2702). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 2710 may be followed by fully connected layers 2712. The fully connected layers 2712 may comprise one or more layers each with one or more neurons that receives an input from a previous layer (e.g., a convolutional or pooling layer) and provides an output to a subsequent layer (e.g., the output layer 2708). The fully connected layers 2712 may be described as “dense” because each of the neurons in a given layer may receive an input from each neuron in a previous layer and provide an output to each neuron in a subsequent layer. The fully connected layers 2712 may be followed by an output layer 2708 that provides the output of the convolutional neural network. The output may be, for example, an indication of which class, from a set of classes, the input 2702 (or any portion of the input 2702) belongs to. The convolutional neural network may be trained using a stochastic gradient descent type algorithm or another suitable algorithm. The convolutional neural network may continue to be trained until the accuracy on a validation set (e.g., a held-out portion from the training data) saturates or using any other suitable criterion or criteria.

It should be appreciated that the convolutional neural network shown in FIG. 27 is only one example implementation and that other implementations may be employed. For example, one or more layers may be added to or removed from the convolutional neural network shown in FIG. 27. Additional example layers that may be added to the convolutional neural network include: a pad layer, a concatenate layer, and an upscale layer. An upscale layer may be configured to upsample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output. As another example, in some embodiments, one or more convolutional, transpose convolutional, pooling, unpooling layers, and/or batch normalization may be included in the convolutional neural network. As yet another example, the architecture may include one or more layers to perform a nonlinear transformation between pairs of adjacent layers. The non-linear transformation may be a rectified linear unit (ReLU) transformation, a sigmoid, and/or any other suitable type of non-linear transformation, as aspects of the technology described herein are not limited in this respect.

Convolutional neural networks may be employed to perform any of a variety of functions described herein. It should be appreciated that more than one convolutional neural network may be employed to make predictions in some embodiments. Any suitable optimization technique may be used for estimating neural network parameters from training data. For example, one or more of the following optimization techniques may be used: stochastic gradient descent (SGD), mini-batch gradient descent, momentum SGD, Nesterov accelerated gradient, Adagrad, Adadelta, RMSprop, Adaptive Moment Estimation (Adam), AdaMax, Nesterov-accelerated Adaptive Moment Estimation (Nadam), AMSGrad.

### Example Computer Architecture

An illustrative implementation of a computer system 2800 that may be used in connection with any of the embodiments of the technology described herein is shown in FIG. 28. The computer system 2800 includes one or more processors 2810 and one or more articles of manufacture that comprise non-transitory computer-readable storage media (e.g., memory 2820 and one or more non-volatile storage media 2830). The processor 2810 may control writing data to and reading data from the memory 2820 and the non-volatile storage device 2830 in any suitable manner, as the aspects of the technology described herein are not limited in this respect. To perform any of the functionality described herein, the processor 2810 may execute one or more processor-executable instructions stored in one or more non-transitory computer-readable storage media (e.g., the memory 2820), which may serve as non-transitory computer-readable storage media storing processor-executable instructions for execution by the processor 2810.

Computing device 2800 may also include a network input/output (I/O) interface 2840 via which the computing device may communicate with other computing devices (e.g., over a network), and may also include one or more user I/O interfaces 2850, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The embodiments described herein can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described herein can be generically considered as one or more controllers that control the functions discussed herein. The one or more controllers can be implemented in numerous ways, such as with dedicated hardware, or with general purpose hardware (e.g., one or more processors) that is programmed using microcode or software to perform the functions recited herein.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the functions discussed herein of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the functions discussed herein, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The terms “program” or “software” are used herein in a generic sense to refer to any type of computer code or set of processor-executable instructions that can be employed to program a computer or other processor to implement various aspects of embodiments as discussed herein. Additionally, it should be appreciated that according to one aspect, one or more computer programs that when executed perform methods of the disclosure provided herein need not reside on a single computer or processor but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing,” “involving,” and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

While some aspects and/or embodiments described herein are described with respect to certain brain conditions, these aspects and/or embodiments may be equally applicable to monitoring and/or treating symptoms for any suitable neurological disorder or brain condition. Any limitations of the embodiments described herein are limitations only of those embodiments and are not limitations of any other embodiments described herein.

What is claimed is:

1. A device wearable by or attached to a person, comprising:
at least one annular array transducer configured to provide ultrasound radiation to perform non-invasive neuromodulation in at least one region of the brain of the person.

2. The device as claimed in claim 1, wherein the device comprises circuitry configured to receive echo data from the annular array transducer and, based on the echo data, correct an amplitude and/or a phase of the ultrasound radiation.

3. The device as claimed in claim 1, wherein the annular array transducer is further configured to provide the ultrasound radiation to perform non-invasive neurostimulation in the at least one region of the brain of the person.

4. The device as claimed in claim 1, wherein the annular array transducer comprises a plurality of concentric elements, wherein at least one of the plurality of concentric elements is operable to provide the ultrasound radiation.

5. The device as claimed in claim 4, wherein each of the plurality of concentric elements have substantially the same surface area.

6. The device as claimed in claim 4, wherein each of the plurality of concentric elements have substantially the same width.

7. The device as claimed in claim 4, wherein the device comprises circuitry configured to independently drive electrical energy to each element of the plurality of concentric elements.

8. The device as claimed in claim 7, wherein a time profile of the electrical energy includes a continuous wave, a quasi-continuous wave, and/or a pulsed wave.

9. The device as claimed in claim 4, wherein the device comprises circuitry configured to assign a phase to each element of the plurality of concentric elements, wherein the phase assigned to the element is independent from phases for other elements.

10. The device as claimed in claim 9, wherein the circuitry is further configured to assign the phase to each element of the plurality of concentric elements based on a distance from the element to a target focal depth in the at least one region of the brain of the person.

11. The device as claimed in claim 10, wherein the circuitry is further configured to adjust the target focal depth by adjusting the phase of one or more elements of the plurality of concentric elements.

12. The device as claimed in claim 1, wherein the annular array transducer is fabricated by radially dicing a piezoelectric material into a plurality of concentric elements.

13. The device as claimed in claim 12, wherein the piezoelectric material is selected such that the piezoelectric material has a minimal lateral mode coupling.

14. The device as claimed in claim 13, wherein the piezoelectric material includes a 1-3 composite, lead metaniobate, a single-crystal piezoelectric material, and/or a composite piezoelectric material.

15. The device as claimed in claim 1, wherein the annular array transducer has a center frequency in a range from 200 kHz to 1 MHz.

16. The device as claimed in claim 1, wherein the annular array transducer has a diameter range from 1 to 4 inches.

17. The device as claimed in claim 1, wherein the annular array transducer has a fractional bandwidth range from 10% to 60% of a center frequency for the annular array transducer.

18. The device as claimed in claim 1, wherein the device includes a processor configured to guide the ultrasound radiation to the at least one region of the brain of the person using ultrasound, optoacoustics, photoacoustics, thermo-acoustics, doppler and functional ultrasound, magnetic resonance-based radiation force imaging (RFI), shear wave elastography, magnetic resonance thermometry, functional imaging techniques, electroencephalography, optical tracking, and/or simulation-based guidance.

19. The device as claimed in claim 1, wherein the annular array transducer comprises a plurality of concentric segments, each segment of the plurality of concentric segments comprising a plurality of elements along a circumference of the segment, wherein at least one of the plurality of elements is operable to provide the ultrasound radiation.

20. The device as claimed in claim 19, wherein the device comprises circuitry configured to independently drive electrical energy to each element of the plurality of elements, of each segment of the plurality of concentric segments.

21. The device as claimed in claim 20, wherein a time profile of the electrical energy includes a continuous wave, a quasi-continuous wave, and/or a pulsed wave.

22. The device as claimed in claim 19, wherein the device comprises circuitry configured to assign a phase to each element of the plurality of elements, of each segment of the plurality of concentric segments, wherein the phase assigned to the element is independent from phases for other elements.

23. The device as claimed in claim 22, wherein the circuitry is further configured to assign the phase to each element of the plurality of elements, of each segment of the plurality of concentric segments, based on a distance from the element to a target focal depth in the at least one region of the brain of the person.

24. The device as claimed in claim 23, wherein the circuitry is configured to adjust the target focal depth in up to three dimensions by adjusting the phase of one or more elements of the plurality of elements of each segment of the plurality of concentric segments.

25. The device as claimed in claim 1, wherein the annular array transducer is fabricated by radially dicing a piezoelectric material into a plurality of concentric segments and circumferentially dicing each segment of the plurality of concentric segments into a plurality of elements.

26. A device wearable by or attached to a person for providing non-invasive neuromodulation or neurostimulation to at least one region of the brain of the person, comprising:
an oscillator;
a phase generator coupled to the oscillator;
a plurality of power amplifiers coupled to the phase generator;
a plurality of tuners, each tuner coupled to a power amplifier of the plurality of power amplifiers;
at least one annular array transducer configured to generate ultrasound radiation, each element of the annular array transducer coupled to a tuner of the plurality of tuners; and
feedback circuitry coupled to the annular array transducer and the phase generator.

27. The device as claimed in claim 26, wherein the feedback circuitry is configured to receive echo data from the annular array transducer and transmit the echo data to the phase generator.

28. The device as claimed in claim 27, wherein the phase generator is configured to correct an amplitude and/or a phase of the ultrasound radiation based on the echo data.

29. The device as claimed in claim 28, wherein the device includes a processor configured to guide the ultrasound radiation to the at least one region of the brain of the person using ultrasound, optoacoustics, photoacoustics, thermo-acoustics, doppler and functional ultrasound, magnetic resonance-based radiation force imaging (RFI), shear wave elastography, magnetic resonance thermometry, functional imaging techniques, electroencephalography, optical tracking, and/or simulation-based guidance.

30. The device as claimed in claim 28, wherein the device includes a processor configured to guide the ultrasound radiation to the at least one region of the brain of the person using portable magnetic resonance imaging having a field strength less than 10 mT, between 10 mT and 0.1 T, or between 0.1 T and 0.2 T.

31. A method of making a device wearable by or attached to a person for providing non-invasive neuromodulation or neurostimulation to at least one region of the brain of the person, comprising:
providing an oscillator;
providing a phase generator coupled to the oscillator;
providing a plurality of power amplifiers coupled to the phase generator;
providing a plurality of tuners, each tuner coupled to a power amplifier of the plurality of power amplifiers;
providing at least one annular array transducer, each element of the annular array transducer coupled to a tuner of the plurality of tuners; and
providing feedback circuitry coupled to the annular array transducer and the phase generator.

32. A method, comprising:
using a device to provide ultrasound radiation to perform non-invasive neuromodulation or neurostimulation in at least one region of the brain of the person,
wherein the device comprises:
an oscillator;
a phase generator coupled to the oscillator;
a plurality of power amplifiers coupled to the phase generator;
a plurality of tuners, each tuner coupled to a power amplifier of the plurality of power amplifiers;
at least one annular array transducer configured to generate the ultrasound radiation, each element of the annular array transducer coupled to a tuner of the plurality of tuners; and
feedback circuitry coupled to the annular array transducer and the phase generator.
